SEQUENCE LISTING (30 sequences)

SEQ ID NO: 1 - DNA, 20 bp, synthetic construct
  source 1..20: mol_type = other DNA
gtaggatgggtagggtggtc

SEQ ID NO: 2 - DNA, 20 bp, synthetic construct
  source 1..20: mol_type = other DNA
agtgatgatgtgtggtaggc

SEQ ID NO: 3 - DNA, 20 bp, synthetic construct
  source 1..20: mol_type = other DNA
agtgtaggtgtggatgatgc

SEQ ID NO: 4 - DNA, 20 bp, synthetic construct
  source 1..20: mol_type = other DNA
taggttttgggtagcgtggt

SEQ ID NO: 5 - DNA, 20 bp, synthetic construct
  source 1..20: mol_type = other DNA
agggatgatgtgtggcagga

SEQ ID NO: 6 - DNA, 20 bp, synthetic construct
  source 1..20: mol_type = other DNA
ctaggacgggtagggcggtg

SEQ ID NO: 7 - DNA, 20 bp, synthetic construct
  source 1..20: mol_type = other DNA
agggatgtgtgtggtaggct

SEQ ID NO: 8 - DNA, 20 bp, synthetic construct
  source 1..20: mol_type = other DNA
agggatgctgcgtggtaggc

SEQ ID NO: 9 - DNA, 20 bp, synthetic construct
  source 1..20: mol_type = other DNA
gagggttggtgtggttggca

SEQ ID NO: 10 - DNA, 20 bp, synthetic construct
  source 1..20: mol_type = other DNA
agggttggtgtgtggttggc

SEQ ID NO: 11 - DNA, 20 bp, synthetic construct
  source 1..20: mol_type = other DNA
atggttggtttatggttggc

SEQ ID NO: 12 - DNA, 20 bp, synthetic construct
  source 1..20: mol_type = other DNA
gaagggtggtcagggtggga

SEQ ID NO: 13 - DNA, 20 bp, synthetic construct
  source 1..20: mol_type = other DNA
ggagggtgggtcgggtggga

SEQ ID NO: 14 - DNA, 20 bp, synthetic construct
  source 1..20: mol_type = other DNA
ggggttggtacagggttggc

SEQ ID NO: 15 - DNA, 20 bp, synthetic construct
  source 1..20: mol_type = other DNA
agatgggcaggttggtgcgg

SEQ ID NO: 16 - DNA, 20 bp, synthetic construct
  source 1..20: mol_type = other DNA
agatgggtgggtagggtggg

SEQ ID NO: 17 - DNA, 20 bp, synthetic construct
  source 1..20: mol_type = other DNA
atagggtgggtgggtgggta

SEQ ID NO: 18 - DNA, 20 bp, synthetic construct
  source 1..20: mol_type = other DNA
tggtggttgggttgggttgg

SEQ ID NO: 19 - DNA, 20 bp, synthetic construct
  source 1..20: mol_type = other DNA
tgggatgggattggtaggcg

SEQ ID NO: 20 - DNA, 20 bp, synthetic construct
  source 1..20: mol_type = other DNA
agggttggttatgtggttgg

SEQ ID NO: 21 - DNA, 20 bp, synthetic construct
  source 1..20: mol_type = other DNA
attggttgggtagggtggtt

SEQ ID NO: 22 - DNA, 20 bp, synthetic construct
  source 1..20: mol_type = other DNA
aaacggttggtgaggttggt

SEQ ID NO: 23 - DNA, 20 bp, synthetic construct
  source 1..20: mol_type = other DNA
cggggtggtgtgggtgggag

SEQ ID NO: 24 - DNA, 20 bp, synthetic construct
  source 1..20: mol_type = other DNA
tattggttggataggttggt

SEQ ID NO: 25 - DNA, 20 bp, synthetic construct
  source 1..20: mol_type = other DNA
agggatgatgtgtggttggc

SEQ ID NO: 26 - DNA, 20 bp, synthetic construct
  source 1..20: mol_type = other DNA
agggatggtgtgtggtaggc

SEQ ID NO: 27 - DNA, 20 bp, synthetic construct
  source 1..20: mol_type = other DNA
agggttgatgtgtggtaggc

SEQ ID NO: 28 - DNA, 20 bp, synthetic construct
  source 1..20: mol_type = other DNA
agggatggtgtgtggttggc

SEQ ID NO: 29 - DNA, 20 bp, synthetic construct
  source 1..20: mol_type = other DNA
agggttgatgtgtggttggc

SEQ ID NO: 30 - DNA, 20 bp, synthetic construct
  source 1..20: mol_type = other DNA
agggttggtgtgtggtaggc

(19) United States
(12) Patent Application Publication
(10) Pub. No.: US 2025/0279163 A1
(43) Pub. Date: Sep. 4, 2025

(54) METHODS, SYSTEMS, AND COMPUTER READABLE MEDIA FOR APTAMER SELECTION

(71) Applicants: ARIZONA BOARD OF REGENTS ON BEHALF OF ARIZONA STATE UNIVERSITY, Scottsdale, AZ (US); Centre national de la recherche scientifique, PARIS (FR); ECOLE NORMALE SUPERIEURE, PARIS (FR); SORBONNE UNIVERSITE, PARIS (FR); UNIVERSITE PARIS CITE, PARIS (FR)

(72) Inventors: Petr Sulc, Scottsdale, AZ (US); Remi Monasson, Paris (FR); Simona Cocco, Paris (FR)

(73) Assignees: ARIZONA BOARD OF REGENTS ON BEHALF OF ARIZONA STATE UNIVERSITY, Scottsdale, AZ (US); Centre national de la recherche scientifique, PARIS (FR); ECOLE NORMALE SUPERIEURE, PARIS (FR); SORBONNE UNIVERSITE, PARIS (FR); UNIVERSITE PARIS CITE, PARIS (FR)

(21) Appl. No.: 18/701,423

(22) PCT Filed: Oct. 14, 2022
(86) PCT No.: PCT/US2022/078104

§ 371 Date: Apr. 15, 2024

Related U.S. Application Data
(60) Provisional application No. 63/256,342, filed on Oct. 15, 2021.

(51) Int. Cl. G16B 40/00; C12N 15/10; C12N 15/115; G06N 3/047; G16B 15/30

(52) U.S. Cl. CPC G16B 40/00; C12N 15/1048; C12N 15/115; G06N 3/047; G16B 15/30; C12N 2310/16; C12N 2320/13

(57) ABSTRACT

Provided herein are methods of generating a trained classifier at least partially using a computer. The methods include training a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model. Additional methods as well as related systems and computer readable media are also provided.

### CROSS-REFERENCE TO RELATED APPLICATONS

This application claims priority to U.S. Provisional Patent Application Ser. No. 63/256,342, filed Oct. 15, 2021, the disclosure of which is incorporated herein by reference.

### BACKGROUND

Discovery and design of molecules that can specifically bind a given target molecule is a key problem in diagnostics, therapeutics and molecular biology in general. Multiple different experimental approaches exist to select specific target binders that include antibodies, short peptide, protein and small molecules. Single stranded oligonucleotides (DNA or RNA) have also been shown to be able to bind with high affinity to a plethora of various targets, including small metabolites, proteins, other nucleic acids, viruses, exosomes, and cells of specific tissue. These short oligonucleotides, called aptamers, are selected from an initial pool of sequences by a procedure known as Systematic Evolution of Ligands by Exponential Enrichment (SELEX). This method consists of multiple rounds of selection, where aptamers that bind strongly enough to the protein target are selected and amplified for the next round, until few strong binders are obtained. The advantages of using DNA or RNA include low cost of synthesising the molecules and relative ease of their manipulation of laboratory setting as opposed to other selection methods such as peptide or antibody selection. Oligonucleotides can be denatured and refolded allowing for multiple selection rounds. On the other hand, as they are composed of four possible types of bases (A, C, G and T/U), they do not offer such chemical diversity as antibodies, which thus limit to some extent the range of targets that they can be selected to bind strongly too. However, chemical modifications of the nucleic bases can increase the chemical space of the aptamers and provide diverse sequence libraries from which strong binders can be selected against variety of targets.

In recent years, machine learning methods have been increasingly often used to process biological sequences datasets, with applications including classifications, binding prediction as well as molecular design. While a significant improvement has recently been achieved in using deep learning for protein or RNA structure predictions, predictions of binding interactions as well as computational design of molecule that can bind to a target molecule remains an outstanding significant challenge.

So far, the prediction of interaction between a small molecule ligand and a target protein has received most attention from the machine learning community, as such approaches are at the basis of drug screening pipeline. More recently, generative models have been used to e.g. design novel viral capsid proteins, based on high-throughput screening of experimental datasets.

Over the last decade, deep neural networks (DNN) have become one of the most popular machine learning tools, which are increasingly more used also in chemical and biological data processing workflow. However, training DNNs typically requires large datasets, which might be challenging to obtain from experiments. DNNs have many free parameters, which makes it challenging to identify and interpret particular features of the molecule are attributed to its ability to bind to a given target. Feature importance and interpretability of DNN predictions is one of the important challenges of machine learning techniques in general. Further challenge presented by biological sequence ensembles is that we often deal with datasets with relative low abundance of available training examples compared to other applications areas for which the DNN architectures been developed, such as image recognition. Finally, presence of errors in the sequence dataset, coming e.g. from error in affinity measurements or sequencing errors, add further difficulties to the training as well as interpretability.

Popular approaches to machine learning on sequences ensembles include inverse problems assembly from statistical physics, such as direct coupling analysis methods, which have been previously successfully used to infer native contacts and guide folding of RNA and proteins based on homologous sequence alignment, as well as to generate functional enzymes based on functional protein alignments and protein-recognizing RNA. They infer correlations between pairs of residues from fields in Potts model, which can be readily interpreted as either a conservation of a single residue or pairwise coupling between distinct residues. More recently, Restricted Boltzmann Machines (RBMs) architectures, a neural network with bipartite graph structure, have been successfully applied as a generative model for protein domain sequences, as well as a predictor of peptides that will be presented on MHC complexes. They present an intermediate between the direct coupling models and DNNs, as they can be trained to recognize multi-residue coupling as opposed to pairwise interactions, but due to limited number of weights between the two neuron layers, the parameters can still be interpreted and rationalized.

Therefore, there is a need for methods, and related aspects, of generating a trained classifier and/or a candidate aptamer.

### SUMMARY

The present disclosure relates, in certain aspects, to methods of generating a trained classifier and/or a candidate aptamer. These and other aspects will be apparent upon a complete review of the present disclosure, including the accompanying figures.

In one aspect, the present disclosure provides a method of generating a trained classifier at least partially using a computer. The method includes training, by the computer, a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model, thereby generating the trained classifier at least partially using the computer.

In some embodiments, the method includes applying a maximum likelihood algorithm to the training dataset to identify and exclude erroneous information. In some embodiments, the training dataset comprises one or more sequence motifs. In some embodiments, the method includes repeating the training step using at least a second training dataset (e.g., with sequence information corresponding to a subsequently selected population of aptamers, and/or one or more descriptors thereof). In some embodiments, the target biomolecule comprises thrombin. Biomolecules other than thrombin can also be targeted using the methods and other aspects disclosed herein. In some embodiments, the method includes generating candidate aptamer sequence information using the trained RBM model. In some embodiments, the method includes synthesizing the candidate aptamer using the candidate aptamer sequence information to produce a synthesized candidate aptamer. In some embodiments, the method includes using the synthesized candidate aptamer to bind the target biomolecule.

In one aspect, the present disclosure provides a method of generating a candidate aptamer. The method includes generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule. The method also includes synthesizing the candidate aptamer using the candidate aptamer sequence information, thereby generating the candidate aptamer.

In one aspect, the present disclosure provides a system, comprising a controller comprising, or capable of accessing, computer readable media comprising non-transitory computer executable instructions which, when executed by at least one electronic processor, perform at least training a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model.

In one aspect, the present disclosure provides a system that includes a biomolecule synthesis device, and at least one controller operably connected to the biomolecule synthesis device, which controller comprises, or is capable of accessing, computer readable media comprising non-transitory computer executable instructions which, when executed by at least one electronic processor, perform at least: generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule; and synthesizing the candidate aptamer using the biomolecule synthesis device and the candidate aptamer sequence information.

In one aspect, the present disclosure provides a computer readable media comprising non-transitory computer executable instruction which, when executed by at least electronic processor perform at least training a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model.

In one aspect, the present disclosure provides a computer readable media comprising non-transitory computer executable instruction which, when executed by an electronic processor perform at least: generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule; and synthesizing a candidate aptamer using an operably connected biomolecule synthesis device and the candidate aptamer sequence information.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1A is a flow chart that schematically shows exemplary method steps of generating a trained classifier according to some aspects disclosed herein.

FIG. 1B is a flow chart that schematically shows exemplary method steps of generating a candidate aptamer according to some aspects disclosed herein.

FIG. 2 is a schematic diagram of an exemplary system suitable for use with some aspects disclosed herein.

FIGS. 3A-3E schematically show the SELEX procedure used to obtain DNA aptamers that bind to thrombin. The procedure consists of the following steps: a) Start with an initial library of DNA sequences. b) DNA aptamers compete with each other to bind to thrombin. c) Sequences that are unbound (or bound too weakly) are washed away. d) Remaining bound sequences dissociate after the sample is heated up. e) Using polymerase chain reaction (PCR), multiple copies are made of the remaining sequences, resulting into a new library of aptamers for the next round of selection.

FIGS. 4A-4D are plots showing that the RBM's results are almost insensitive to noise. Only sequences with 3 counts or more in the original dataset are considered in this figure. (a): comparison of counts inferred by the correction algorithm used and of counts obtained from the SELEX experiment; local maxima have almost always more inferred counts than counts, and sequences with few counts close to sequences with many counts have typically 0 inferred counts (light grey points). (b): distribution of counts of different sequences that the correction algorithm indicates as errors (dashed line) or that cannot be completely explained by errors (black line). (c): Upper panel-log-likelihoods computed by the RBM trained with the K sequences having more than 0 inferred counts (RBM+C), for the full 8-th round dataset (black line) or for sequences tested experimentally (lighter and darker grey vertical bars). Lower panel—same log-likelihoods obtained with the RBM trained on K random sequences (RBM−C). Both RBMs are able to distinguish between good and bad binders obtained experimentally, with a AUROC of 0.98 (RBM+C) and 0.99 (RBM−C). (d): Log-likelihood obtained by RBM+C and RBM−C for the sequences tested experimentally; the Pearson r is 0.93.

FIGS. 5A-5C are plots showing that the RBM's log-likelihood is related to the sequence fitness. (a): Histograms of the log-likelihood of all sequences in the dataset at different rounds, obtained through an RBM trained on the sequences at round 6 (see Methods for details). The black line denotes the average log-likelihood. Each peak observed at round 5 corresponds to functionally differentiated sequences, as can be observed in the right plots, where the conservation logos of all the sequences with likelihoods in the shaded areas are presented. (b): For each round r, the log-likelihood of all sequences in each bin shown (a) is compared with the difference of the logarithm of the number of sequences in the same bin at two consecutive rounds. The linear fits are done by using points with log-likelihood >−45. The inset shows the log-likelihood variance plotted against the difference of average log-likelihood in subsequent rounds. (c): The 10 left and right 20-nucleotide sequences with most counts at the last round are considered, and their number of counts is given here as a function of the round number. The counts are normalized so that all rounds have the same total number of sequences.

FIG. 6 are plots showing that sequences in the two loops are not correlated. Sequence logos: the logos of two RBM's weights are shown, and each weight is focused on a sub-region (loop) of the full aptamer sequence. Histogram: the RBM's weights are normalized so that their Frobenius norm is 1, and the squared Frobenius norm is computed for each loop, so that the two contributions sum to 1. The logarithms of the minimum of the two contributions, computed for each hidden unit, are used to produce the histogram.

FIGS. 7A-7D are plots showing that RBM's weights allow for biological insight and interpretation of the model's performances. (a): the four families identified in [Y. Zhou, X. Qi, Y. Liu, F. Zhang, and H. Yan. Dna-nanoscaffold-assisted selection of femtomolar bivalent human alpha-thrombin aptamers with potent anticoagulant activity. ChemBioChem, 20 (19): 2494-2503, 2019] can be separated in different clusters when each sequence is projected into the space obtained by the inputs to two specific hidden units (HU) of the RBM. (b): position of the sequences experimentally tested in the same input space of panel (a). (c): sequences binding the thrombin in the AB exosite and in the CD exosite are both at high log-likelihood, but in most cases correspond to different values of the input to the HU 28. (d): some weights of the RBM trained on the 8th-round dataset are shown. HU 1 and HU 8 can be used to cluster the four families identified in [Y. Zhou, X. Qi, Y. Liu, F. Zhang, and H. Yan. Dna-nanoscaffold-assisted selection of femtomolar bivalent human alpha-thrombin aptamers with potent anticoagulant activity. ChemBioChem, 20 (19): 2494-2503, 2019] (see panel (a)), and HU 28 can distinguish between sequences binding to the AB and CD exosite.

FIG. 8 is a plot showing that The RBM can predict aptamer-thrombin binding. The RBM used here has been trained on all the (unique) sequences observed in the last round of SELEX, and the corresponding log-likelihood histogram is given as a black line. The lighter and darker grey vertical lines correspond to the log-likelihood of the sequences experimentally validated for binding with thrombin (Table 1). The threshold (black dashed line) has been fixed by the prediction performed on a preliminary set of sequences.

FIG. 9 is a 5% Native gel assay @ 15° C. of stem loops (1-23) alone in the presence of Mg²⁺/K⁺ (lane 1) and allowed to mix with α-thrombin for 30 min @ 25° C. on the bench (lane 2).

FIG. 10A-10B show gel images of: a) a binding site assay of all binding sequences in the RBM generated dataset. Lane 1 shows addition of thA and lane 2 shows addition of thD to the thrombin pre-incubated strand (labeled in black). All sequences shown here bind exosite CD except for an7 which binds to exosite AB. b) a binding site assay of the 6 sequences that make up the sequence space between thA and test sequence an9. Lane 1 shows addition of thA and lane 2 shows addition of thD to the thrombin pre-incubated strand (labeled in black). All sequences with point mutation (A→T) at nucleotide 17 (p11, p21, p22) show binding to exosite CD while those without (p12, p13, p23) show binding to exosite AB.

FIG. 11 provides plots showing that RBMs trained on sequences from different rounds are similar. The off-diagonal (grey) plots show the correlation of the log-likelihoods computed on all observed 40 nt-aptamers (in all rounds) with all possible pairs of RBMs (only 10000 points randomly chosen are shown). Notice that, if Eq. (7) describes the dynamics of the system and the initial distribution is uniform, the correlation between RBMs at different rounds should give a Pearson coefficient of 1 in the ideal case. The diagonal (spots) plots show the histogram of the log-likelihoods of the same set of sequences, computed with each RBM.

FIG. 12 provides plots showing that RBM log-likelihood is correlated with the Levenshtein distance with respect to ThD. For each sequence observed in round 8, the log-likelihood given by the RBM is plotted here against the Levenshtein distance with respect to the ThD aptamer (GTAGGATGGGTAGGGTGGTC (SEQ ID NO: 1), star), which has been experimentally checked to bind the thrombin with an extremely low dissociation constant. The plots on the left side and below the two-dimensional histogram are histograms giving the marginalization with respect to the variable in the other axis. Crosses and circles represent the functional aptamers experimentally tested in this work.

FIG. 13 is a plot showing the setting of the binding threshold for the RBM log-likelihood binding. The RBM used here has been trained on all the (unique) sequences observed in the last round of SELEX, and the corresponding log-likelihood histogram is given as a black line. The lighter and darker grey vertical lines correspond to the log-likelihood of the sequences experimentally validated for binding with thrombin in a preliminary experiment and with the known binders from [Y. Zhou, X. Qi, Y. Liu, F. Zhang, and H. Yan. Dna-nanoscaffold-assisted selection of femtomolar bivalent human alpha-thrombin aptamers with potent anticoagulant activity. ChemBioChem, 20 (19): 2494-2503, 2019] (ThA, ThB, ThC and ThD), and the threshold (black dashed line) has been fixed as the mean log-likelihood value which allows for the best discrimination.

FIG. 14 are plots showing that sequences at later rounds tend to have good log-likelihood for the left or right part. The RBM used here has been trained on all the (unique) sequences observed in the last round of SELEX. For each full sequence (40 nts) observed in a given round, the corresponding left and right sub-sequences are taken and the RBM is used to compute the log-likelihood. The dashed line are guides for the eyes, to show that the number of sequences in the bottom left part of each plot decreases with successive rounds. The number of sequences found in this region is significantly lower than what would be expected under a random pairing of left and right sequences (p-value lower than 0.001).

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Classifier. As used herein, “classifier” generally refers to algorithm computer code that receives, as input, test data and produces, as output, a classification of the input data as belonging to one or another class.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.”

Modified Nucleotide: As used herein, “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-methoxyethoxy, 2′-fluoro, 2′-allyl, 2′-O-[2-(methylamino)-2-oxoethyl], 4′-thio, 4′-CH2-O-2′-bridge, 4′-(CH2)2-O-2′-bridge, 2′-LNA or other bicyclic or “bridged” nucleoside analog, and 2′-O—(N-methylcarbamate) or those comprising base analogs. In connection with 2′-modified nucleotides as described for the present disclosure, by “amino” is meant 2′—NH2 or 2′-O—NH2, which can be modified or unmodified.

Nucleic Acid Aptamer: As used herein, “nucleic acid aptamer” refers to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target, and facilitating the reaction between the target and another molecule.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

System: As used herein, “system” in the context of medical or scientific instrumentation refers a group of objects and/or devices that form a network for performing a desired objective.

Threshold: As used herein, “threshold” refers to a separately determined value used to characterize or classify experimentally determined values.

### DETAILED DESCRIPTION

In some embodiments, RBMs to a set of DNA aptamer sequences obtained from our prior work using SELEX method. We develop a maximum likelihood algorithm for curation of the sequences in order to account for high rate of sequencing errors that is present in the SELEX dataset. We then show that the trained RBM model's sequence likelihood can be directly linked to the fitness of the particular sequence during the selection. RBM model that is trained on an earlier round of the selection is able to predict fitness of sequences in the next rounds. We further show that we can identify the sequence motif that corresponds to the largest likelihood of an aptamer, and we use the RBM to generate new sequences that have not been part of the experimental dataset. We experimentally verify the RBM prediction of good or bad binding properties for these new generated sequences. Finally, we compare the RBM model performance with some of the popular DNN architectures and show that while DNNs have problem generalizing on our experimental dataset, the RBM models perform well as both classifiers and generators.

In some embodiments, Restricted Boltzmann Machine (RBM) architectures are trained on the DNA aptamer datasets generated from SELEX experiments. We developed a procedure to curate datasets (assuming a given sequencing error rate in the ensemble) to make them more suitable for use as a training dataset. We further enforce a sparse connection between the hidden layer and the input layer, where the sequences are presented. This way, we show that the RBM is able to learn the representation of the good binders, and we show that the score assigned by the RBM to the sequence is directly related to its fitness in the experimental procedure. Not only that this algorithm can be used to assess good and bad binders, but also it is able to be used as a generative model. To experimentally validate the model, we generated 23 sequences from the model, and 21 of them behaved as expected based on the prediction of the model (14 of them were generated as binders and indeed all of them were experimentally verified to indeed bind to the protein. These sequences were not part of the training ensemble. 9 sequences were generated to be non-binders, by breaking a few as possible bases in the original strong binding sequence). Finally, our model is interpretable: due to relatively small number of parameters compared to deep networks, it is possible to identify sequence motifs that are contributing the most to the sequence to bind to its target.

To illustrate, 1A is a flow chart that schematically shows exemplary method steps of generating a trained classifier. As shown, method 100 includes training a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model (step 102).

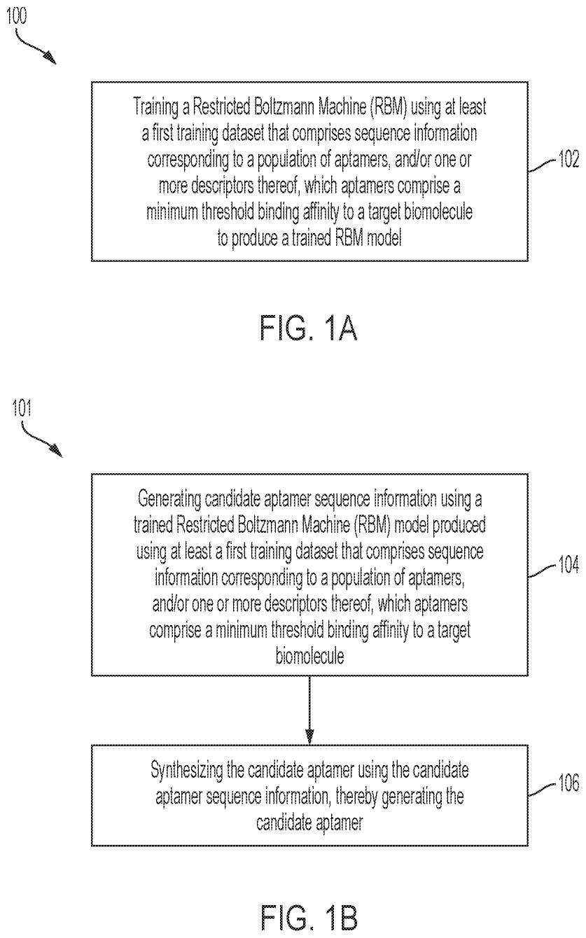

To further illustrate, FIG. 1B is a flow chart that schematically shows exemplary method steps of generating a candidate aptamer. As shown, method 101 includes generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule (step 104). Method 101 also includes synthesizing the candidate aptamer using the candidate aptamer sequence information (step 106).

In some embodiments, the method includes applying a maximum likelihood algorithm to the training dataset to identify and exclude erroneous information. In some embodiments, the training dataset comprises one or more sequence motifs. In some embodiments, the method includes repeating the training step using at least a second training dataset (e.g., with sequence information corresponding to a subsequently selected population of aptamers, and/or one or more descriptors thereof). In some embodiments, the target biomolecule comprises thrombin. In some embodiments, the method includes generating candidate aptamer sequence information using the trained RBM model. In some embodiments, the method includes synthesizing the candidate aptamer using the candidate aptamer sequence information to produce a synthesized candidate aptamer. In some embodiments, the method includes using the synthesized candidate aptamer to bind the target biomolecule.

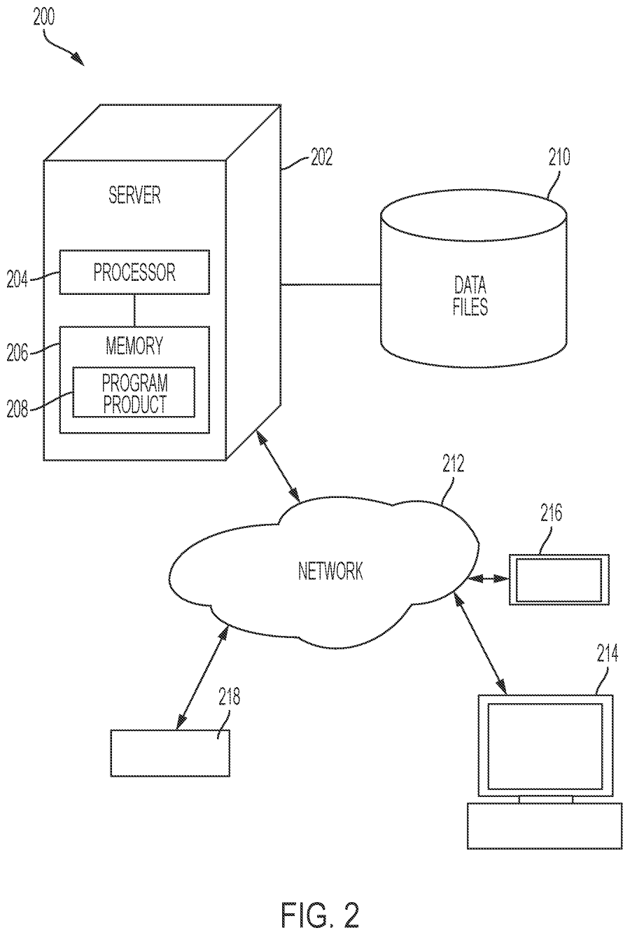

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server), which includes processor 204 and memory, storage device, or memory component 206, and one or more other communication devices 214, 216, (e.g., client-side computer terminals, telephones, tablets, laptops, other mobile devices, etc. (e.g., for receiving subject data sets, etc.) in communication with the remote server 202, through electronic communication network 212, such as the Internet or other internetwork. Communication devices 214, 216 typically include an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 200 also includes program product 208 (e.g., for detecting generating a trained classifier and/or generating a candidate aptamer as described herein) stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., entries corresponding to temporal and spatial data, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication devices 214, 216 in these aspects, for example, can be a laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 208 includes non-transitory computer-executable instructions which, when executed by electronic processor 204, perform at least: generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule, and synthesizing (e.g., via biomolecule synthesis device 218) the candidate aptamer using the biomolecule synthesis device and the candidate aptamer sequence information.

### Example: Generative and Interpretable Machine Learning for DNA Aptamer Design and Analysis

### 1. Introduction

This example applies the RBMs to a set of DNA aptamer sequences obtained from our prior work using SELEX method [Y. Zhou, X. Qi, Y. Liu, F. Zhang, and H. Yan. Dna-nanoscaffold-assisted selection of femtomolar bivalent human alpha-thrombin aptamers with potent anticoagulant activity. ChemBioChem, 20 (19): 2494-2503, 2019]. We develop a maximum likelihood algorithm for curation of the sequences in order to account for sequencing errors that are present in the SELEX dataset. We then show that the trained RBM model's sequence likelihood can be directly linked to the fitness of the particular sequence during the selection. RBM model that is trained on an earlier round of the selection is able to predict fitness of sequences in the next rounds. We further show that we can identify the sequence motif that corresponds to the largest likelihood of an aptamer, and we use the RBM to generate new sequences that have not been part of the experimental dataset. We experimentally verify the RBM prediction of good or bad binding properties for these new generated sequences. Finally, we compare the RBM model performance with some of the popular DNN architectures and show that while DNNs have problem generalizing on our experimental dataset, the RBM models perform well as both classifiers and generators.

### 2. Results and Discussion

2.1 Dataset Obtained from SELEX Procedure

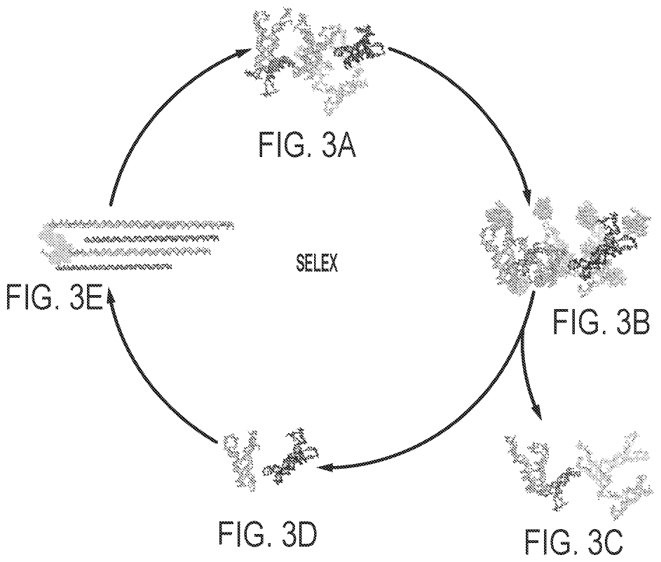

We obtained the dataset used here for training in our prior work [Y. Zhou, X. Qi, Y. Liu, F. Zhang, and H. Yan. Dna-nanoscaffold-assisted selection of femtomolar bivalent human alpha-thrombin aptamers with potent anticoagulant activity. ChemBioChem, 20 (19): 2494-2503, 2019], which used SELEX method to obtain a bivalent DNA nanostructure that binds to a thrombin protein. In the typical DNA SELEX procedure, we start with an initial library of 10¹⁵ unique DNA sequences all around the same length. The library is then exposed to the target tethered to surface. The non-binding sequences are then washed away. The binding sequences are then collected (and optionally also sequenced) and amplified using PCR where they serve as the sequence library for the next cycle of SELEX. The cycle is repeated until binders of the desired binding affinity are found. The washing intensity is increased in later rounds to obtain stronger binders. In the particular experimental dataset used, the SELEX procedure was performed on a DNA nanotile (FIG. 3), consisting of a joined-double helix region with two loops of length 20 nucleotides each. While the helix structure was conserved across all DNA structures, the two respective loops were variable, starting from initial random library. The SELEX procedure is schematically shown in FIG. 3 and consisted of eight selection rounds. The binding structures were sequenced in rounds 5, 6, 7 and 8. For each round, our dataset includes the sequence of the two (left and right) respective variable loop regions of the DNA nanotile, as well as the number of counts of the respective DNA loops sequence, corresponding to the the number of times it was found in the sequencing dataset. In the typical SELEX protocols, the sequences with the largest number of counts in the last rounds are considered the best binders.

### 2.2 RBM and Training

### 2.2.1 Probabilistic Model

A Restricted Boltzmann Machine (RBM) is a probabilistic model, which assigns a probability to a system state, composed by two parts: a visible one (which, in our case, is the aptamer), v=(v₁, . . . , v_{N}), and an “hidden” one, h=(h₁, . . . , h_{M}). The probability is

$$p ( v , h ) = \frac{1}{Z} exp ( {\sum }_{i = 1}^{N} {g}_{i} ( {v}_{i} ) - {\sum }_{μ = 1}^{M} {𝒰}_{u} ( {h}_{μ} ) + {\sum }_{μ , i} {h}_{μ} {w}_{μ i} ( {v}_{i} ) ) , \qquad ( 1 )$$

where Z is the normalization, gᵢ, and w_{μi} are parameters to be learned by using the data, and

$${𝒰}_{μ} = \frac{1}{2} {{γ}_{μ +} ( {h}_{+} )}^{2} + \frac{1}{2} {{γ}_{μ -} ( {h}_{-} )}^{2} + {θ}_{μ +} {h}_{+} + {θ}_{μ -} {h}_{-} , \qquad ( 2 )$$

where h₊=max (h, 0), h₋=min (h, 0) and γ_{μ+}, γ_{μ-}, θ_{μ+}, θ_{μ-} are parameters to be inferred from the data. This specific form of the potential U_{μ}, which is called “double Rectified Linear Unit”, has proved to be an effective choice, which combines the usage of a relatively low number of parameters with the possibility of learning high-order correlations in the data.

### 2.2.2 Training

The training of an RBM consists in finding the parameters so that the log-likelihood of the observed data, that is

$$ℒ = {\sum }_{s} log p ( s ) = {\sum }_{s} \int dh log p ( s , h ) , \qquad ( 3 )$$

is maximized. Here the sum over s encompasses all the sequences observed through sequencing of the SELEX experiment. Notice that this maximization is done after marginalizing over the hidden units, since these are not given as observed data. The maximization of L is in general a difficult problem, but several effective techniques to obtain good parameter values have been developed so far, for instance contrastive divergence and persistent contrastive divergence. In this work, we used the latter method to train the machine.

As in many neural-network training schemes, an hyperparameter concerning the regularization can be added. It has been suggested that, for RBMs, a large regularization on the weight parameters, together with a high number of hidden units, can improve the generative properties of the machine and the interpretability of the weights learned. In particular, here we used a L₁² regularization scheme, which consists in adding to the log-likelihood a term of the form

$$- λ {\sum }_{μ} {( {\sum }_{i , υ} \vert {w}_{μ i \vert} )}^{2} , \qquad ( 4 )$$

which is specifically designed to increase the weight sparsity, and has been used in previous works. The value of the hyperparameter A has been fixed so that it is as large as possible without decreasing in a relevant way the log-likelihood of the training set. We also validated several choices of this hyperparameter through a validation set of aptamers obtained in a preliminary experiment, as explained below.

In this work, we used the RBM to learn the probability distribution of a large set of (potential) aptamers obtained after a SELEX procedure. This specific problem presents several additional challenges that must be addressed in the training of the model. The first choice consists in deciding the number of visible units used, that can be set to 40, that is twice the length of the single aptamer to model the possibility of bivalent aptamers explicitly, or 20, that is the length of a single aptamer. The choice here depends on which phenomena we want our RBM to capture: the SELEX evolution or the binding capability. In the first case, an RBM with 40 visible units may be more suitable, as a double aptamer with both loops able to bind thrombin is expected to have a fitness advantage with respect to an aptamer with a single good loop. For the second case, it is known that 20 nucleotides are enough to bind thrombin in both exosites, so an RBM with 20 visible units will be better at characterizing good and bad binders. The second challenge related to our specific dataset is the presence of information regarding the (relative) abundances of 40-nt aptamers (counts in the sequenced data). This problem has a very little effect for 40-nucleotide aptamers since only 0.8% of the reads are repetitions in the last round, while it is more relevant when dealing with 20-nucleotide aptamers, since in this case, putting together aptamers observed in the left and right loop, the repetitions accounts for 73.6% of the reads in the last round. In this last case, we decided not to include the information about counts in the RBM training, because this is not a genuine indicator of the binding affinity. Indeed, we expect biases introduced by the specific experimental condition (e. g. PCR), and the presence of “hitchhiking” sequences, which end up being over-represented because the partner aptamer (other side of the 40-nucleotide sequence) is a good binder. Moreover, we validated this by using RBMs to distinguish binders from non-binders in a preliminary experiment and observed slightly better results obtained with the RBM trained without using information about read counts in the training set.

### 2.2.3 Sampling

The RBM is a generative model, that is once the parameters in Eq. (1) are obtained, we can sample this probability distribution and obtain new sequences. Under the hypothesis that the RBM log-likelihood of a sequence is related to its fitness in the experiment (or its property of being a good or bad binder), as discussed below, we can focus on high-log-likelihood sequences to sample probable good binders or low-log-likelihood sequences to sample probable bad binders. In particular, the sampling of likely good binders can be done by sampling the following probability distribution

$${\tilde{p}}_{β} ( υ ) = {( \int dh p ( υ , h ) )}^{β} , \qquad ( 5 )$$

with β>1. The sampling of probable bad binders, on the opposite, is particularly interesting when the sequence obtained is close, in Hamming distance, to a good binder. This can be achieved by considering a good binder (i.e. sequence experimentally checked to be able to bind thrombin) and checking if some among the neighbouring sequences have low log-likelihood.

Remarkably, the sampling of new sequences is a particularly hard task for the dataset used here, as long as we focus on finding new, potent aptamers. Indeed, consider an initial library of N DNA sequences of length L. Assuming that the initial library is randomly created, the probability of a given sequence of not being in the initial library is

$${( 1 - {4}^{- L} )}^{N} ≃ {e}^{- {4}^{- L} N} . \qquad ( 6 )$$

For L=40 and N=10¹⁵ this quantity is almost 1, that is the vast majority of the possible double aptamers are not present in the original library. However, if we focus on single aptamers, we have L=20 and so the probability given above is almost zero (in particular, it is ≈10⁻³⁴⁵), and so it is extremely unlikely that the initial database does not explore exhaustively the full set of possible single aptamers. Therefore, in the ideal SELEX experiment, we should not be able to find any aptamer not already observed at the end of the evolution which is able to strongly bind thrombin.
2.2.4 Robustness with Respect to Reading Errors

A common drawback of next-generation sequencing techniques is the inevitable presence of sequencing errors. The error rate, that is the probability of recording a wrong nucleotide instead of the correct one, can reach 0.1, giving rise, in our case, to 2 errors on average for each single aptamer obtained. In principle, this plethora of errors could prevent the RBM from learning genuine patterns, so we tested the robustness of RBMs and we checked that the results we obtained are insensitive to the presence of sequencing errors, as long as the error rate is low enough not to completely delete the signal present in the data.

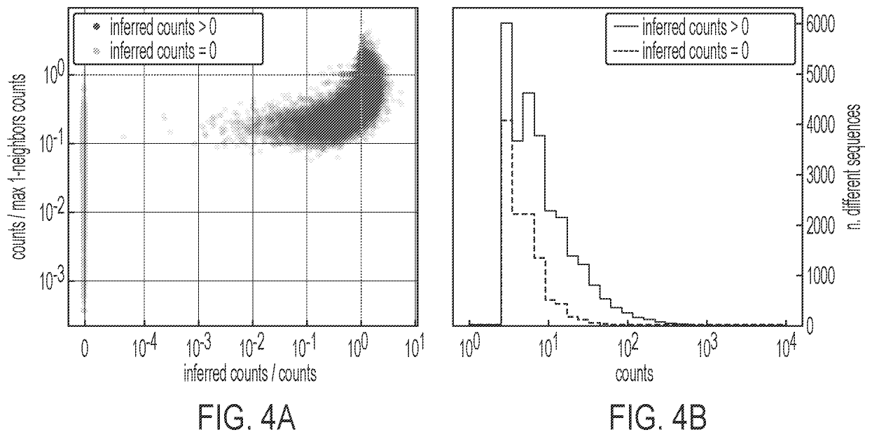

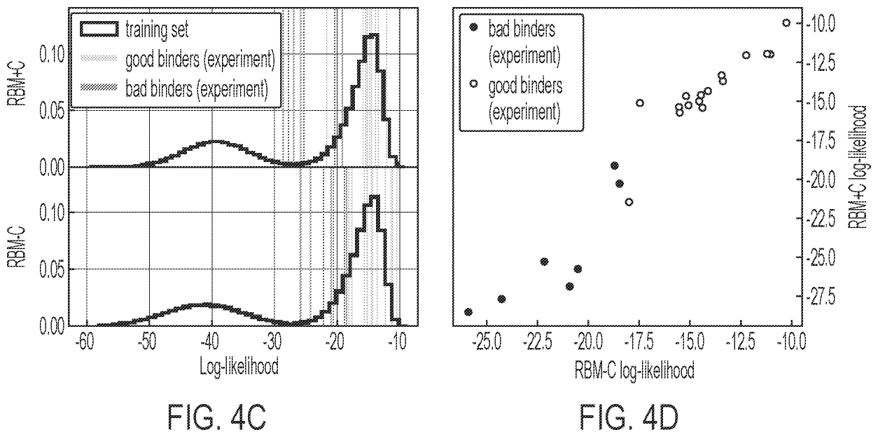

Our test, presented in FIG. 4, consists in training the RBM with the sequences observed in the left and right loops in the last round of selection, before or after using an error-correction algorithm designed to reduce sequencing errors in the data. This algorithm, which is based on a maximum-likelihood approach, uses the counts of the sequence together with the local topology of the dataset to infer the most likely number of counts (under the error model and the hypothesis described in Sec. 4.2) that each sequence had in the real sequence dataset, without the sequencing errors. This number of “inferred counts” can be in turn used, by setting a threshold, to decide which sequences are likely to be in the dataset only because of sequencing errors. The details of the algorithm are given in Sec. 4.2, and we show how this algorithm, when used as a classificator to discard likely spurious sequences, outperforms simpler methods such as a count-based rejection approach.

In FIGS. 4a and 4b we show the effect of the algorithm on the dataset, focusing on the specific case where an error is called whenever the inferred number of counts outputted by the algorithm is equal to zero. In FIGS. 4c and 4d we trained two RBMs respectively on the dataset of the K=46835 sequences with more than zero inferred counts, and on the dataset obtained by keeping the K sequences with the highest number of counts. The results, both in terms of the log-likelihood given to the training set and of the performance in discriminating good from bad binders in experiments, are very similar, confirming the remarkable robustness of the RBM with respect to the presence of sequencing errors. As a further test, we also obtained very similar results when the RBM is trained on K randomly extracted sequences (among the sequences with 3 or more counts).

By comparing the RBMs trained on these subsets of sequences with high number of counts (or inferred counts) with the RBMs trained on the full set of sequences without any attempt to correct the sequencing errors, we observed that training the machine with a much larger number of sequences gives slightly better performances (as measured on a validation set of sequences) with respect to the training with a much lower number of sequences which are more likely to be correct. Therefore, in the following, we will use the full set of sequences as training set for the RBMs.

2.3 the Log-Likelihood Inferred by the RBM is Compatible with the Sequence Fitness

Strongly functional aptamers can be obtained by starting with a large library of random RNA or DNA molecules and performing selection through several rounds according to the biomolecules' ability to bind a specific target. In the ideal case, the population of aptamers in such an experiment is described by the recursion equation

$$p ( g , r + 1 ) = \frac{{e}^{{α}_{r} F ( g )}}{{〈 {e}^{{α}_{r} F ( g )} 〉}_{r}} p ( g , r ) , \qquad ( 7 )$$

where p(g, r) is the probability of observing the aptamer g at round r, [symbol] f [symbol] , =Σgp(g, r)f(g) denotes the average at round r, αᵣ is a parameter which tunes the selection strength to go from round r to r+1 and, finally, F(g) is the fitness of aptamer g. The fitness is related to the aptamer's capability of binding its target, so it does not depend on the round number. Therefore we have

$$p ( g , r ) = \frac{1}{𝒩} exp [ ( {\sum }_{i = 1}^{r - 1} {α}_{i} ) F ( g ) + log ( {p}_{0} ( g ) ) ] , \qquad ( 8 )$$

where N is the normalization, and p₀(g) is the probability distribution of the initial library. As described in the Methods section, the RBM's parameters are trained so that the probability of the observed data according to the RBM model is maximized. Under the hypothesis that the initial library is uniformly sampled at random, this is guaranteed to happen if minus the energy −E(g) inferred through the RBM is equal to the fitness. However, even for large datasets it is difficult to understand a priori whether a choice of the RBM's parameters exists such that the RBM's energy can be identical (or even close) to the “ground-truth” fitness.

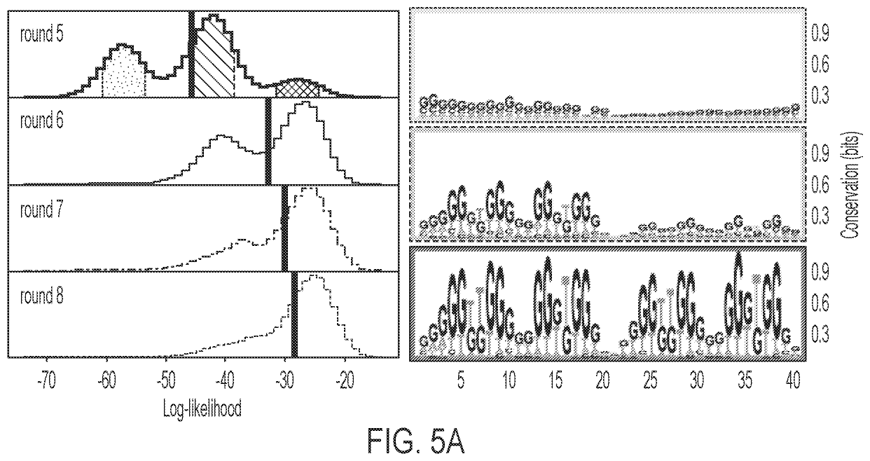

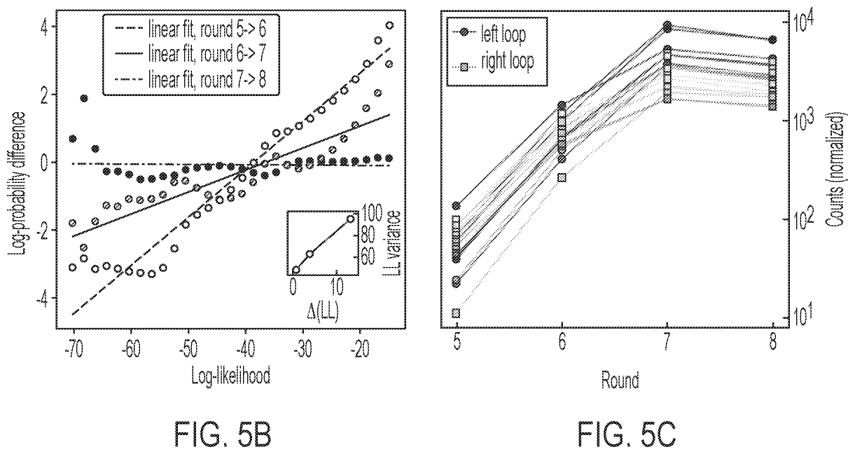

The relationship between log-likelihood inferred through the RBM and the fitness can be investigated by looking at the evolution of the sequences in subsequent rounds. An RBM trained with the double-loop aptamer sequences observed in round 5 assigns log-likelihood to the round 5 aptamers as shown in FIG. 5a. Three peaks are apparent, and the logos of the sequences with corresponding log-likelihood are presented: the peak for low log-likelihoods is formed by many non-specialized sequences, whose logo simply shows an abundance of G nucleotides in both loops; the intermediate peak, instead, shows a structured left loop, with a clearly visible G-quadruplex motif; finally, the little peak made of sequences with high log-likelihood is obtained when both loops are structured in a similar way, with again clearly visible G-quadruplex structures. Remarkably, when the same RBM is used to compute the log-likelihoods of the sequences in the following rounds, the peaks at low and intermediate log-likelihood shrink as the sequences accumulate in the peak at high log-likelihood. Accordingly, the average log-likelihood (indicated by a black bar in the figure) increases with rounds.

The fact that sequences at high log-likelihood are more likely to be present at later rounds strongly suggests a positive covariance of the log-likelihood and the fitness. Moreover, using Eq. (7), we can check to what extent a linear relationship between these two quantities seems reasonable. Indeed, under the hypothesis that aptamers with different fitness have different log-likelihood, from Eq. (7) we have that the probability p(s, r) of observing an aptamer with log-likelihood s at round r satisfies

$$\frac{p ( s , r + 1 )}{p ( s , r )} = \frac{{e}^{{α}_{r} ℱ ( s )}}{{〈 {e}^{{α}_{r} ℱ ( s )} 〉}_{r}} , \qquad ( 9 )$$

where F(s) is the (unique) fitness associated at log-likelihood s, and [symbol] f [symbol] r=Σs p(s, r)f(s) denotes the average at round r. The probabilities p(s, r) are obtained by the histograms in FIG. 5a, then the logarithm of the left hand side of Eq. (9) is plotted against the average log-likelihood of each histogram bar in FIG. 5b. The linear relationship results in a good fit (once we restrict ourselves to aptamers with log-likelihood greater than −45 to avoid dealing with strong finite size effects), confirming once more the presence of a striking similarity between log-likelihood and fitness. Notice that the slopes of the fitting lines decrease with the number of rounds, and this could be motivated by a decrease in selection strength at later rounds. This hypothesis is supported by the fact that the 10 most common sequences present in the last round for each loop do not increase exponentially in the rounds considered here, as shown in FIG. 5c.

### 2.4 the RBM Unveils Lack of Correlations Between the Two Loops

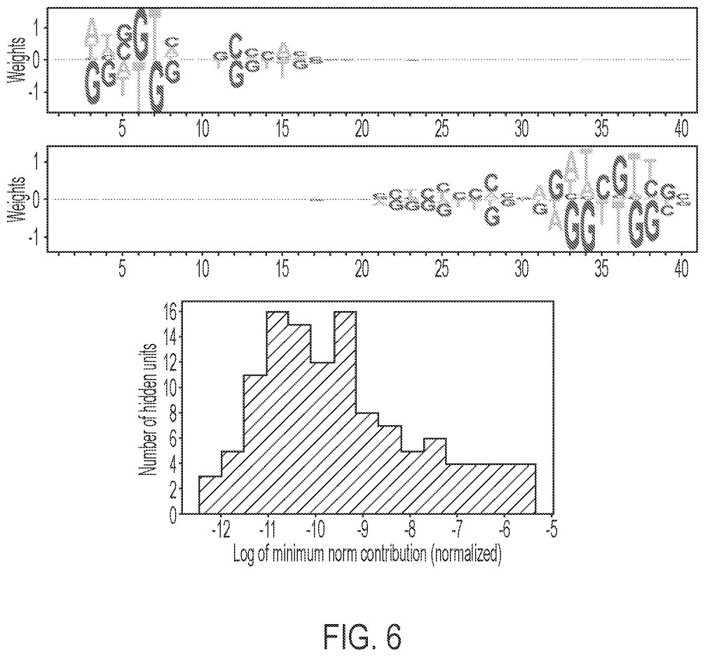

In the SELEX data analyzed here, the two loops can potentially bind the thrombin independently, giving higher selection probability to aptamers presenting both loops able to strongly bind the target. And indeed, as apparent from FIG. 5a, toward the end of the training most sequences have both loops showing a strong conservation of a G-quadruplex structure. However, the question of whether there are correlations between the two loops cannot be answered by looking at the sequence logos presented in FIG. 5a. To discover the presence of correlations, however, we can try to visually inspect the weights learned by the RBM after the training is complete. The two weights with the largest Frobenius norm for the RBM trained on the last round aptamers are shown in FIG. 6, and it is apparent that no correlations are observed, through these two weights, between the first 20 nucleotides (left loop) and the last 20 nucleotides (right loop). Moreover, a similar pattern can be observed in all other weights, which are conserved either on the left or on the right loop. Indeed, we collected all the squared Frobenius norms of the left and right loops, after the full Frobenius norm of each weight has been normalized to 1. Then for each weight we took the minimum between these two quantities, and we build with these values the histogram in FIG. 6. Since the maximum of this values is <e⁻⁵, the RBM is not capturing any relevant correlation between the two loops. Notice that in [Y. Zhou, X. Qi, Y. Liu, F. Zhang, and H. Yan. Dna-nanoscaffold-assisted selection of femtomolar bivalent human alpha-thrombin aptamers with potent anticoagulant activity. ChemBioChem, 20 (19): 2494-2503, 2019] the authors observed a certain level of allosteric linkage. However, they needed additional competition assays to observe this effect, and we suggest that this cannot be observed directly from the SELEX data they collected.

### 2.5 Generative Model of New Binders

The RBM is a generative model, and as such it can be used to sample new aptamer sequences. In particular, since we showed that sequences with high log-likelihood also have high fitness in the original SELEX experiment, we can use the RBM log-likelihood as a proxy for binding affinity to thrombin and attempt generating sequences which should be able to bind thrombin. We sampled from the RBM 10 sequences with high log-likelihood that were not present in the original dataset (named an9 to an17 and an22, an23 in Table 1), and we experimentally checked their binding with thrombin, see Sec. 2.6. All of them turned out to be correctly predicted as sequences able to bind thrombin. Notice that, it is unlikely that good aptamers are not found by this SELEX experiment but, due to subsampling effects, reading errors or experimental biases this can still be the case, as it happens here. However, all of the sequences obtained here are at Hamming distance 1 or 2 from the original dataset, and we did not find any sequence with high log-likelihood which has a larger distance from the dataset. We also tried to use our RBM to make the lowest possible number of mutations to decrease the log-likelihood of known good binders. In particular, sequence an1 has 1 mutation from a control sequence that we tested for binding, an2 has 1 mutation of difference from thB (tested in [Y. Zhou, X. Qi, Y. Liu, F. Zhang, and H. Yan. Dna-nanoscaffold-assisted selection of femtomolar bivalent human alpha-thrombin aptamers with potent anticoagulant activity. ChemBioChem, 20 (19): 2494-2503, 2019]), an3 has 1 mutation of difference from thC (tested in [40]). All of them were confirmed not being anymore able to bind thrombin after this single-point mutation. However, all these mutations removed a G from the sequence, and it is well known that G nucleotides are necessary to form G-quadruplexes which in turn stabilize the 3D structure of the aptamer allowing a stronger interaction with thrombin. To show that also other positions and other nucleotides are relevant, we also designed two more sequences, an4 and an5, which have 2 mutations with respect to two aptamers that were found in the SELEX database and that we validated as good binders (the same used for an1 and an3). In this case, the mutations are chosen so that the log-likelihood is decreased, but without removing G nucleotides from the original sequences. Also in this case, the sequences turned out to lose the ability of binding thrombin after the 2 mutations, as predicted.

Finally, we tried to push the RBM prediction capabilities to their limit by searching for sequences with high number of counts (139 or more) and low log-likelihood and sequences with low number of counts (11 or less) and high log-likelihood. The sequences chosen are an6, an7, an18, an19 and an8, an9, an20, an21 in the second set, and the RBM prediction were confirmed in all cases but one (an18), although all the sequences of the first set were found to be very close to the threshold that we used to make predictions (that has been fixed with a preliminary experiment), and hence we expect the predictions to be less accurate in this region of log-likelihood.

TABLE 1
RBM generated sequences with log-likelihood from the RBM trained on the
(unique) sequences observed in the last round. Thrombin binding prediction (from
the trained RBM, using the threshold obtained from a preliminary experiment) and
results (from gel shift assay) are labeled (B) for binder and (NB) for non-binder.

| | | SEQ | | | |
|---|---|---|---|---|---|
| | | ID | Log- | Binding | |
| Label | Sequence | NO: | likelihood | Prediction | Binding Result |
| an1 | AGTGATGATGTGTGGTAGGC | 2 | −23.41 | NB | NB |
| an2 | AGTGTAGGTGTGGATGATGC | 3 | −24.02 | NB | NB |
| an3 | TAGGTTTTGGGTAGCGTGGT | 4 | −22.28 | NB | NB |
| an4 | AGGGATGATGTGTGGCAGGA | 5 | −23.54 | NB | NB |
| an5 | CTAGGACGGGTAGGGCGGTG | 6 | −21.15 | NB | NB |
| an6 | AGGGATGTGTGTGGTAGGCT | 7 | −23.91 | NB | NB |
| an7 | AGGGATGCTGCGTGGTAGGC | 8 | −20.00 | B | B |
| an8 | GAGGGTTGGTGTGGTTGGCA | 9 | −10.99 | B | B |
| an9 | AGGGTTGGTGTGTGGTTGGC | 10 | −11.77 | B | B |
| an10 | ATGGTTGGTTTATGGTTGGC | 11 | −14.71 | B | B |
| an11 | GAAGGGTGGTCAGGGTGGGA | 12 | −15.64 | B | B |
| an12 | GGAGGGTGGGTCGGGTGGGA | 13 | −14.97 | B | B |
| an13 | GGGGTTGGTACAGGGTTGGC | 14 | −14.91 | B | B |
| an14 | AGATGGGCAGGTTGGTGCGG | 15 | −16.25 | B | B |
| an15 | AGATGGGTGGGTAGGGTGGG | 16 | −14.31 | B | B |
| an16 | ATAGGGTGGGTGGGTGGGTA | 17 | −14.96 | B | B |
| an17 | TGGTGGTTGGGTTGGGTTGG | 18 | −12.29 | B | B |
| an18 | TGGGATGGGATTGGTAGGCG | 19 | −20.36 | B | NB |
| an19 | AGGGTTGGTTATGTGGTTGG | 20 | −20.00 | B | B |
| an20 | ATTGGTTGGGTAGGGTGGTT | 21 | −12.21 | B | B |
| an21 | AAACGGTTGGTGAGGTTGGT | 22 | −12.35 | B | B |
| an22 | CGGGGTGGTGTGGGTGGGAG | 23 | −14.70 | B | B |
| an23 | TATTGGTTGGATAGGTTGGT | 24 | −13.07 | B | B |

### 2.6 Experimental Verification

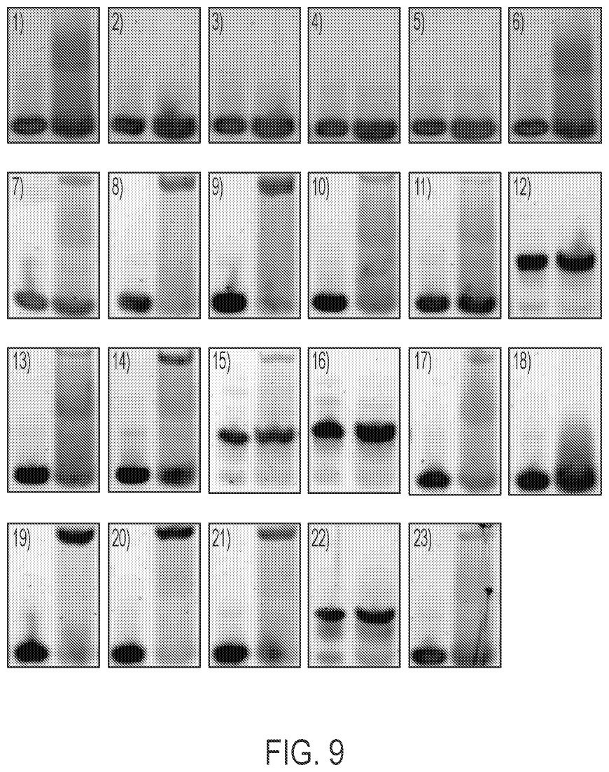

All RBM designed sequences were first assessed for their ability to bind either of the cationic exosites of human alpha-thrombin. Each sequence was placed as the loop of a 18 bp stem loop. As done previously [Y. Zhou, X. Qi, Y. Liu, F. Zhang, and H. Yan. Dna-nanoscaffold-assisted selection of femtomolar bivalent human alpha-thrombin aptamers with potent anticoagulant activity. ChemBioChem, 20 (19): 2494-2503, 2019], we used a 5% native gel shift assay to qualitatively assess the binding of each stem loop to thrombin. Each sequence was tested with two gel lanes, the first lane always corresponding to the stem loop without thrombin and the second lane consisting of equimolar (5 μM) amounts of thrombin and the stem loop. The presence of an upper band, consisting of a stem loop bound to thrombin complex, in the second lane indicates a binding sequence. Sequences without the upper band (nonbinding sequences) either very weakly interact with thrombin, characterized by a smear but no band in the second lane, or don't interact with thrombin at all matching their negative control lane. Sequences ThA and ThD were selected from the previous study as positive controls for their high affinity for thrombin and known binding site. Results for all RBM generated sequences are shown in FIG. 9 and summarized in Table 1.

A secondary band prominently appeared among four of the sequences during the binding assay, (an12, an15, an16, and an22). Upon further investigation, the secondary band was found to be a dimer state between G-quartet motifs in the loop. The higher G-content (relative to the other RBM sequences) and the clear shift from the monomer state in 1×TAE Mg²⁺ (no K⁺) buffer to the dimer state upon addition of buffer with K⁺ provides strong evidence of the dimer G-quartet state.

Presence of the dimer state interfered with binding of the stem-loop to thrombin. However, due to the requirement of K⁺ to form the loop/protein complex, the dimer state could not be avoided, only disrupted. Before addition of the thrombin with its 1×PBS Mg²⁺/K⁺ buffer, the stem-loop samples were remade in 1×TAE Mg²⁺ and heated to 90° C. for 5 min before being immediately chilled in ice. The resulting gel showed binding of the stem-loop for all dimer dominant sequences at the original concentration and conditions. Accordingly, we classify these sequences as binders and suggest their absence from the original dataset is due to G-quartet dimer formation during the original SELEX procedure.

### 2.7 Exosite Binding Location

All binding sequences, aside from the those with strong dimer states, shown in Table 2, were tested against known aptamers ThA and ThD from the original dataset to determine the exosite of thrombin which they bind. ThA is known to bind exosite I and ThD binds exosite II [Y. Zhou, X. Qi, Y. Liu, F. Zhang, and H. Yan. Dna-nanoscaffold-assisted selection of femtomolar bivalent human alpha-thrombin aptamers with potent anticoagulant activity. ChemBioChem, 20 (19): 2494-2503, 2019]. Using fluorophore labeled ThA and ThD sequences, we preincubated each test sequence with thrombin for 30 min @ 25° C.

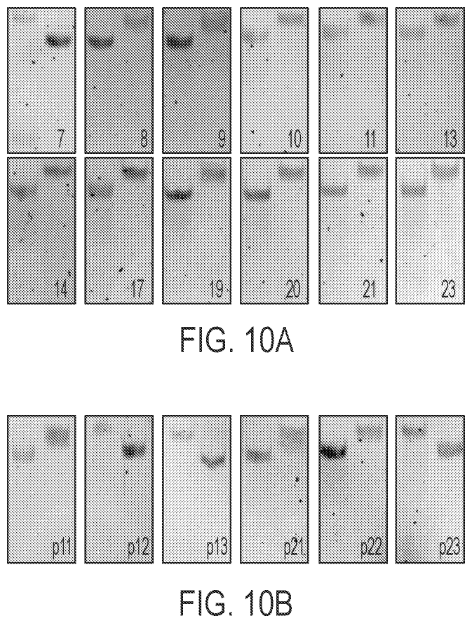

Small amounts (1:10 test sequence) of ThA and ThD were added shortly before gel electrophoresis at the same conditions of the binding assay. By observing the response of both fluorophore labeled control strands shown in FIG. 10, we can determine whether the test strand competes for exosite I or II. In all cases, cooperative binding of ThA or ThD and the test sequence caused a downward shift of the protein/stem loops complex band. While the downward shift of the stem loop/thrombin complex might seem unexpected due to the additional mass of a second stem loop, we show that the thrombin/DNA interaction is occurring in a 1:1 ratio for both a single stem loop binding thrombin and two stem loops cooperatively binding thrombin. Competition for the same exosite was indicated by both a decreased absorbance of the fluorophore control strand relative to a negative control of the fluorophore strand alone binding thrombin and cooperative binding observed from the other fluorophore labeled control strand. The gel results are shown in FIG. 10 and summarized in Table 2.

TABLE 2
Exosite prediction (from RBM) and results (from binding−site assays) for
all binding sequences excluding dimer−susceptible sequences (an12, an15,
an16, an22).

| | | SEQ ID | Exocite | |
|---|---|---|---|---|
| Label | Sequence | NO: | Prediction | Exocite Result |
| an7 | AGGGATGCTGCGTGGTAGGC | 8 | AB | AB |
| an8 | GAGGGTTGGTGTGGTTGGCA | 9 | CD | CD |
| an9 | AGGGTTGGTGTGTGGTTGGC | 10 | CD | CD |
| an10 | ATGGTTGGTTTATGGTTGGC | 11 | CD | CD |
| an11 | GAAGGGTGGTCAGGGTGGGA | 12 | AB | CD |
| an13 | GGGGTTGGTACAGGGTTGGC | 14 | CD | CD |
| an14 | AGATGGGCAGGTTGGTGCGG | 15 | AB | CD |
| an17 | TGGTGGTTGGGTTGGGTTGG | 18 | CD | CD |
| an19 | AGGGTTGGTTATGTGGTTGG | 20 | CD | CD |
| an20 | ATTGGTTGGGTAGGGTGGTT | 21 | CD | CD |
| an21 | AAACGGTTGGTGAGGTTGGT | 22 | CD | CD |
| an23 | TATTGGTTGGATAGGTTGGT | 24 | CD | CD |
| p11 | AGGGATGATGTGTGGTTGGC | 25 | CD | CD |
| p12 | AGGGATGGTGTGTGGTAGGC | 26 | AB | AB |
| p13 | AGGGTTGATGTGTGGTAGGC | 27 | AB | AB |
| p21 | AGGGATGGTGTGTGGTTGGC | 28 | CD | CD |
| p22 | AGGGTTGATGTGTGGTTGGC | 29 | CD | CD |
| p23 | AGGGTTGGTGTGTGGTAGGC | 30 | AB | AB |

Further analysis of our results, indicated a 3 mutation pathway from the highest binding thA sequence (binds exosite I) to a exosite II binding sequence (test sequence an9). Testing all six possible intermediate sequences, labeled as p11-p23 in Table 2, a single point mutation which changes thA into a CD binding sequence was identified. A change in the 17th nucleotide of the loop sequence from Adenine to Thymine caused the change in exosite binding.

2.8 Comparison with Other Machine Learning Approaches

We further compare the RBM model with other popular ML models that have been previously used on DNA sequences. In particular, we train ResNet, Siamese Network and Variational Autoencoder deep neural network (DNN) learning architectures on our aptamer dataset. Furthermore, we include comparison with a single decision tree, a random forest and a gradient boosted classification tree. We describe below how we prepare the training ensemble. We optimize the hyperparameters of each DNN with two different metrics and quantify their performance on the validation subset and the experimentally generated datasets from Table 1.

### 2.8.1 Datasets

Starting with the raw SELEX data, we have both 20 nt aptamer sequences in each arm of the DNA scaffold and a copy number, representing the number of times that sequence was observed during sequencing.

One particular challenge with any next-gen sequencing information is the error rate for misread bases. For each base read using an Illumina MiSeq instrument (as done for our dataset), there's approximately a 0.5±0.9% chance the base is incorrectly identified. These errors complicate analysis of any sequencing info. To help combat this, sequences with hamming distance greater than 1 from any other sequence and with a known good binder on the other arm were trimmed from the dataset.

Three datasets were generated from the trimmed sequences: sequences from the left arm (L), sequences from the right arm (R), and sequences from both arms (B). Each dataset consists of all good binders (copy number >1) and 15 randomly sampled bad binders (copy number=1) per good binder. Training sets (80% of good binders, 35 k sequences for L and R, 70 k for B) and validation sets (20% of good binders, 12 k sequences for L and R, 25 k for B) were split from the dataset. As a further verification dataset we used the experimental results from both the RBM generated sequences as well as DCA generated sequences. All sequences were one-hot encoded prior to training, validation, or prediction.

As using only sequences from the 8th round introduces a general bias of all sequences interacting with thrombin, three more datasets were generated (GL, GR, and GB) with good binders selected as previously done but bad binders were randomly sampled from a set of generated random sequences outside the SELEX dataset's sequence space. The generated datasets were the same sizes as those mentioned previously (L, R, B).

### 2.8.2 Model Selection

For the classification task we used 5 different deep learning models: 2 versions of a Variational Auto Encoder, 2 versions of a Resnet and a Siamese Network Model. Additionally we used 3 classic Machine Learning methods: a decision tree, a random forest and a gradient boosted tree classifier to also classify the sequences as binders or non-binders.

To compare performance of our DNN models, we assessed the accuracy of each model to predict a binder/nonbinder label for each experimentally validated dataset: the RBM generated dataset and the DCA experimental dataset. We also calculated the F1 score metric by comparison of each model's prediction with the ground truth. The F1 score is the harmonic mean of precision, the number of true positives divided by the sum of true positives and false positives, and recall, the number of true positives divided by the sum of true positives and false negatives, in a binary classification task. A F1 score was calculated for each dataset and a mean F1 score was determined by weighting each individual F1 score by the number of total sequences in the dataset. Scores for the DNN models are provided in Table 3.

TABLE 3
Accuracy Scores for all models trained on the Left Arm (L), Right Arm (R) Both Arms (B), Generated Left
Arm (GL), Generated Right Arm (GR) or Generated Both Arms (GB) datsets. Validation sets were taken
as 10% of the training data, while experimental sets consisted of the 23 experimental sequences.

| | Validation | Experimental | DCA | F1 | | Validation | Experimental | DCA | F1 |
|---|---|---|---|---|---|---|---|---|---|
| Model | Acc. | Acc. | Acc. | mean | Model | Acc. | Acc. | Acc. | mean |
| AHSA Resnet Long | | | | | Bayes Resnet Long | | | | |
| L | 0.792 | 0. [symbol] 48 | 0.7 [symbol] | 0.443 | L | 0.304 | 0. [symbol] 95 | 0.31 [symbol] | 0. [symbol] 48 |
| R | 0.7 [symbol] | 0. [symbol] 48 | 0.5 [symbol] | 0. [symbol] 08 | R | 0.381 | 0. [symbol] 2 | 0.31 [symbol] | 0. [symbol] 3 |
| B | 0.75 [symbol] | 0. [symbol] | 0. [symbol] | 0.598 | B | 0.280 | 0. [symbol] 9 [symbol] | 0.31 [symbol] | 0. [symbol] 48 |
| GL | 0.099 | 0.739 | 0.3 [symbol] | 0.071 | | | | | |
| GR | 0.099 | 0.8 [symbol] 9 | 0. [symbol] | 0.7 [symbol] 1 | | | | | |
| GB | 0.098 | 0.73 [symbol] | 0.438 | 0.0 [symbol] 4 | | | | | |
| AHSA Resnet Short | | | | | Bayes Resnet Short | | | | |
| L | 0.7 [symbol] 8 | 0. [symbol] | 0. [symbol] 88 | 0.5 [symbol] | L | 0. [symbol] | 0. [symbol] | 0. [symbol] | 0.648 |
| R | 0.7 [symbol] 8 | 0. [symbol] | 0.7 [symbol] 0 | 0.583 | R | 0.7 [symbol] 6 | 0.478 | 0. [symbol] 8 | 0.542 |
| B | 0.7 [symbol] 7 | 0.43 [symbol] | 0.875 | 0.657 | B | 0.738 | 0. [symbol] | 0. [symbol] 25 | 0. [symbol] |
| GL | 0. [symbol] 98 | 0.739 | 0.438 | 0.684 | | | | | |
| GR | 0. [symbol] | 0.7 [symbol] | 0.438 | 0.684 | | | | | |
| GB | 0. [symbol] | 0.826 | 0. [symbol] 43 | 0.739 | | | | | |
| AHSA VAE Long | | | | | Bayes VAE Long | | | | |
| L | 0.708 | 0. [symbol] 04 | 0. [symbol] | 0.000 | L | 0. [symbol] | 0.304 | 0.688 | 0.000 |
| R | 0. [symbol] | 0. [symbol] 8 | 0.3 [symbol] | 0. [symbol] | R | 0. [symbol] 4 | 0.304 | 0.688 | 0.000 |
| B | 0.47 [symbol] | 0. [symbol] | 0.3 [symbol] | 0. [symbol] 78 | B | 0. [symbol] | 0.304 | 0.688 | 0.000 |
| GL | 0.834 | 0.304 | 0. [symbol] 88 | 0.000 | | | | | |
| GR | 0.82 [symbol] | 0.304 | 0. [symbol] 88 | 0.000 | | | | | |
| GB | 0.840 | 0.304 | 0. [symbol] 88 | 0.000 | | | | | |
| AHSA VAE Short | | | | | Bayes VAE Short | | | | |
| L | 0.7 [symbol] | 0.478 | 0. [symbol] 5 | 0. [symbol] 52 | L | 0. [symbol] 6 | 0.304 | 0. [symbol] | 0.000 |
| R | 0.729 | 0. [symbol] | 0. [symbol] | 0.000 | R | 0. [symbol] 81 | 0.304 | 0. [symbol] | 0.000 |
| B | 0. [symbol] 42 | 0. [symbol] | 0. [symbol] | 0.000 | B | 0. [symbol] 2 | 0.304 | 0. [symbol] | 0.000 |
| GL | 0.997 | 0.82 [symbol] | 0. [symbol] | 0.777 | | | | | |
| GR | 0.994 | 0. [symbol] | 0.500 | 0.724 | | | | | |
| GB | 0.999 | 0. [symbol] | 0.4 [symbol] | 0.713 | | | | | |
| PBT Si [symbol] | | | | | | | | | |
| L | 0.687 | 0. [symbol] | 0.6 [symbol] 2 | 0.2 [symbol] 5 | | | | | |
| R | 0. [symbol] | 0. [symbol] | 0. [symbol] | 0.342 | | | | | |
| B | 0. [symbol] 43 | 0. [symbol] 30 | 0.5 [symbol] 7 | 0.3 [symbol] | | | | | |

[symbol]

indicates data missing or illegible when filed

During training of DNN (L, R, B) models, they never achieved greater than 79.2% accuracy on the validation dataset during training. These same models performed much worse than the RBM on prediction of the RBM generated sequences, with the highest accuracy score of 69.6% (16/23) achieved by the ASHA VAE long, Bayes Resnet Long, and Bayes Resnet short models.

DNN models trained on datasets with generated bad binders (GL, GR, and GB) unsurprisingly performed very well on their validation datasets with all models except for the ASHA Long VAE achieving greater than a 99% accuracy. On average, these models outperformed their counterparts in binding prediction of the RBM generated sequences with the highest accuracy of 86.9% (20/23) achieved by the ASHA Resnet Long. The clear difference in predictive accuracy between models trained on (L, R, B) vs (GL, GR, GB) we believe to be due to the (GL, GR, GB) DNN models learning to look for a consensus good binder, most likely having a G-quartet pattern as the bad binder examples used for training did not. Despite their improved performance on the RBM generated dataset, their performance on the DCA set showed decreased accuracy indicating an incomplete representation.

Errors in sequence data arising from subsampling and next gen sequencing contribute to the difficulty of any DNN model to generalize to the dataset. The small number of sequences, relative to the size of datasets typically used to train DNN models, also contribute to their poor performance.

### 2.8.3 Traditional ML Results

Our traditional ML techniques' performance was measured by the same metrics as our DNN models, namely the accuracy on the RBM generated sequences, the accuracy on the DCA generated sequences, and the F1 mean of both datasets. The single decision tree trained on dataset (GB) obtained the highest accuracy score on the generated RBM set of all non-RBM models with 91.3% (21/23) on the RBM generated dataset. It's sibling model, a single tree trained on dataset (GR), achieved the highest accuracy on the DCA 81.3% (13/16) on the DCA generated dataset the best mean F1 score of 0.838.

TABLE 4
Accuracy Scores for single tree, random forest and gradient boosted
forest trained on the Left (L), Right (R), Both (B), Generated Left
Arm (GL), Generated Right Arm (GR) or Generated Both Arms (GB) datasets.
Validation sets were taken as 20% of the training data, while the experimental
dataset consisted of the 23 experimental sequences.

| Model | Validation Acc. | Experimental Acc. | DCA Acc. | F1 Mean |
|---|---|---|---|---|
| Single Tree | | | | |
| L | 0.116 | 0.739 | 0.313 | 0.692 |
| R | 0.122 | 0.696 | 0.313 | 0.696 |
| B | 0.111 | 0.739 | 0.375 | 0.739 |
| GL | 0.999 | 0.783 | 0.813 | 0.803 |
| GR | 0.999 | 0.869 | 0.813 | 0.838 |
| GB | 0.885 | 0.913 | 0.625 | 0.789 |
| Random Forest | | | | |
| L | 0.242 | 0.609 | 0.438 | 0.654 |
| R | 0.270 | 0.609 | 0.313 | 0.642 |
| B | 0.294 | 0.565 | 0.313 | 0.621 |
| GL | 0.940 | 0.696 | 0.375 | 0.666 |
| GR | 0.931 | 0.739 | 0.375 | 0.679 |
| GB | 0.932 | 0.739 | 0.438 | 0.690 |
| Gradient Boosted Forest | | | | |
| L | 0.098 | 0.695 | 0.313 | 0.679 |
| R | 0.097 | 0.695 | 0.313 | 0.679 |
| B | 0.098 | 0.695 | 0.313 | 0.679 |
| GL | 0.091 | 0.695 | 0.313 | 0.679 |
| GR | 0.091 | 0.695 | 0.313 | 0.679 |
| GB | 0.167 | 0.695 | 0.313 | 0.679 |

This is in stark contrast to our gradient boosted classification tree which performed poorly on every dataset no matter the hyperparameters tried. The random forest performed worse than the single tree, on average performing about as well as most DNN models.

### 3 Conclusion

We have showed that Restricted Boltzmann Machines can be used as a classifiers as well as generators on a set of aptamer sequences obtained from a SELEX experiment. The RBM sequence likelihood was shown to correlate with sequence fitness in a population genetics model of the sequence selection in the experiment. While the RBM model was shown to be able to generalize on the experimental dataset, it has less free parameters than DNNs and allows for identification which activation pattern on the input layer contributes the most to the high sequence likelihood. A G-rich sequence was found to be the most contributing to the high likelihood score from the RBM. The motif is indicative of a G-quartet group, a known functional motif in the DNA aptamers that bind thrombin. We have generated 23 new aptamer sequences from the RBM that were either predicted to bind or not bind to thrombin. Out of 14 sequences that were generated as binders, all 14 were indeed found to bind to thrombin, and out of 9 sequences generated as non-binders, 7 did not bind thrombin in experiment. When generating the non-binders, we aimed to change as few nucleotides as possible to break the binding ability. We next studied the ability of the RBM to generate a better binder than the best obtained from the experiment. Even though our model was able to generate a sequence with higher likelihood than the best binder obtained from the original SELEX dataset, experiment comparing the generated and original best binder showed that the RBM did not generate binders that would have higher affinity then the ones obtained from the experiment, as the size of the initial aptamer library for the SELEX experiment likely covered large part of the possible solutions.

In summary, the RBM model has proved to be trainable on datasets obtained from evolutionary selection experiment, and were shown to be able to act both as classifiers as well as generate novel binders, with the possibility to identify sequence motifs that contribute the most to the high scores. We anticipate that RBMs will be also useful for analysis of other available aptamer datasets, including competition assays where RNAs are selected to bind to a desired target (e.g. cancerous tissues) and at the same time not bind to the control (healthy tissue), with the possibility of obtaining better binders for these complex targets, as well as classify motifs that are enriched or avoided in the final dataset. The RBMs can also help to design experimental protocols to e.g. select the optimal number of rounds needed before no further improvement of aptamer binding is predicted. While we developed here the RBM modeling framework in the context of SELEX protocols, our approach is readily applicable to other selection-amplification protocols, such as phage display for antibody discovery, which have much larger space of possible sequences (20^{L} for sequence of length L) compared to aptamers (4^{L}). We found that the RBM model has performed better on our SELEX dataset than several tested DNN architectures. It is possible to develop DNN models specifically for sequence ensembles from selection experiment, where the DNNs will have an RBM-based input layer that can well capture the functional features in the sequences.

### 4 Methods

### 4.1 RBM Training

We trained several RBMs which have been used for the analysis presented here. In all cases, we followed the general principle of using a quite large number of hidden units, with a quite strong regularization to avoid overfitting. For each RBM trained, the hyperparameters have been fixed by maximizing the log-likelihood of a training and a validation set, so that the number of parameters is not too large to allow for interpretation. For the full range of parameter explored we never saw signs of overfitting (i.e. decrease of the log-likelihood of the validation set together with an increase of the log-likelihood of the training set), and we motivated this with the very large datasets that are available for training the machine. Indeed, this remained true even when we completely turned off any regularization. In all the RBMs used here the only regularization used has been a “L²” regularization scheme, which consists in adding to the log-likelihood a term of the form

$$- λ {\sum }_{μ} {( {\sum }_{i , υ} \vert {w}_{μ i \vert} )}^{2} . \qquad ( 10 )$$

Another hyperparameter that we set is the number of monte-carlo steps done for each update of the parameters, which has been fixed after fixing the number of hidden units and the regularization, by choosing the highest more monte-carlo steps which maximizes the log-likelihood of the validation set. However, we observed that all the results are insensitive to this choice.

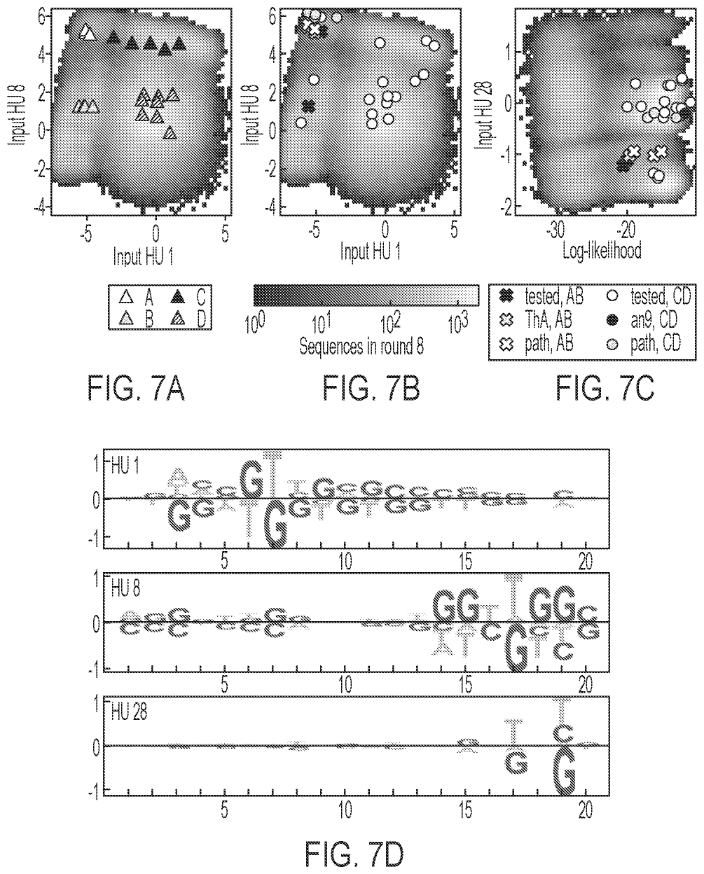

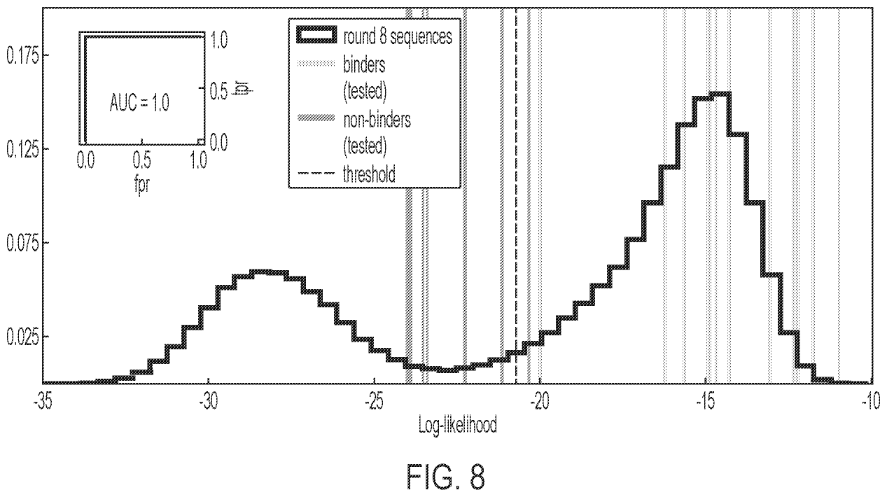

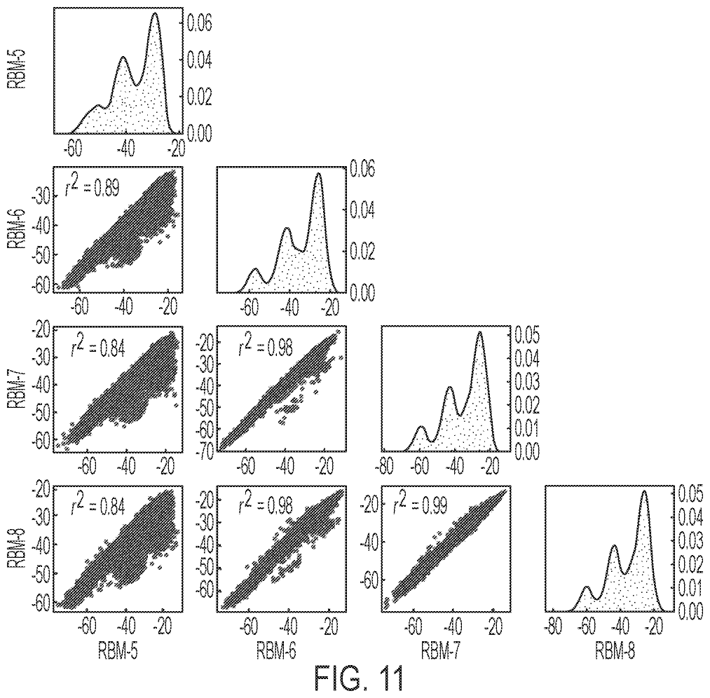

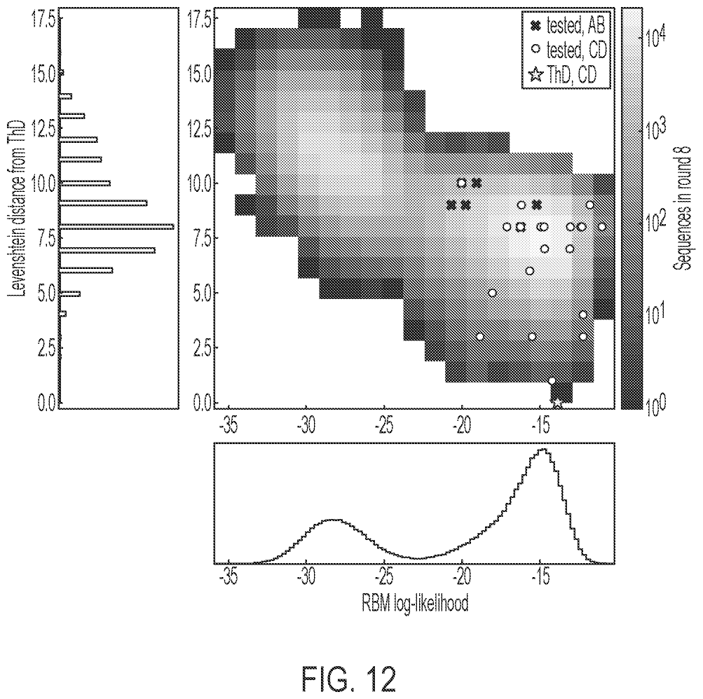

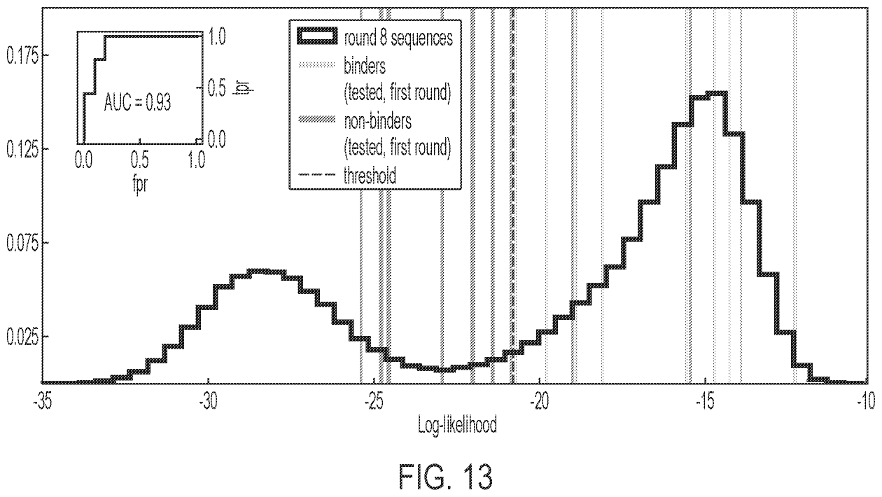

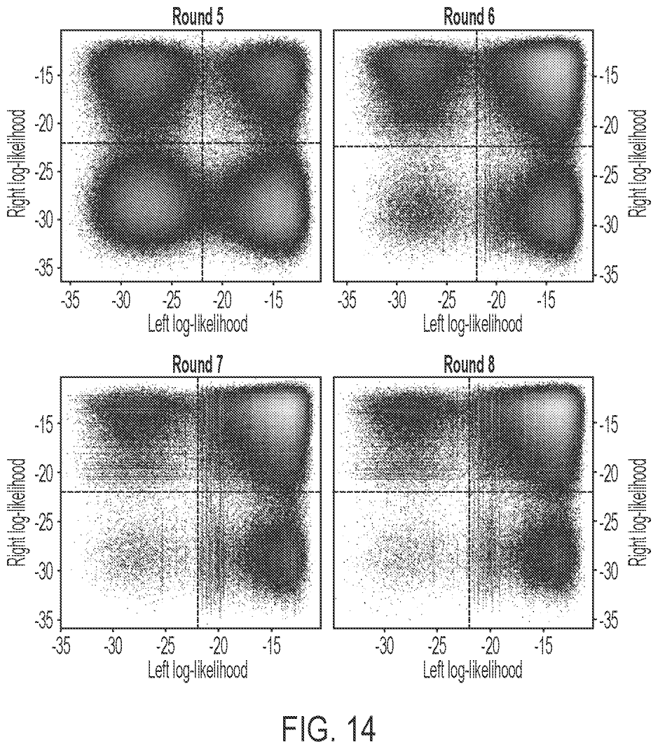

For each training, we used 90% of the dataset as training set and 10% of the dataset as validation set to check that no overfitting is observed. The training set was divided in mini-batches and for each epoch each mini-batch was used to perform an update of the parameters, using the persistent contrastive divergence algorithm. In all cases the training stopped after 20000 updates of the RBM parameters. The size of the mini-batches has been fixed so that the full training is made of at least 20 epochs. In particular, we used the following RBMs in this example:
RBM-D5 (FIG. 11), trained on the double aptamers (40 nucleotides) obtained from the SELEX 5th round. Multiple copies of the same aptamer are neglected during training. The parameters are: 40 visible units, 90 hidden units, λ₁₂=0.01, 8 monte-carlo steps for each update of the parameters, mini-batches of size 800.
RBM-D6 (FIGS. 5, 6 and 11), trained on the double aptamers (40 nucleotides) obtained from the SELEX 6th round. Multiple copies of the same aptamer are neglected during training. The parameters are: 40 visible units, 90 hidden units, λ₁₂=0.01, 10 monte-carlo steps for each update of the parameters, mini-batches of size 500.
RBM-D7 (FIG. 11), trained on the double aptamers (40 nucleotides) obtained from the SELEX 7th round. Multiple copies of the same aptamer are neglected during training. The parameters are: 40 visible units, 110 hidden units, λ₁₂=0.01, 4 monte-carlo steps for each update of the parameters, mini-batches of size 800.
RBM-D8 (FIG. 11), trained on the double aptamers (40 nucleotides) obtained from the SELEX 8th round. Multiple copies of the same aptamer are neglected during training. The parameters are: 40 visible units, 110 hidden units, λ₁₂=0.01, 2 monte-carlo steps for each update of the parameters, mini-batches of size 800.
RBM-S (FIGS. 7, 8, 12, 13, 14 and Tables 1, 2), trained on the single aptamers (20 nucleotides) obtained from the SELEX 8th round, merging sequences from the left and right loops. Multiple copies of the same aptamer are neglected. The parameters are: 20 visible units, 70 hidden units, λ₁₂=0.01, 4 monte-carlo steps for each update of the parameters, mini-batches of size 500.
RBM-S−C (FIG. 4), trained on the single aptamers (20 nucleotides) obtained from the SELEX 8th round, neglecting sequences with less than 3 counts and then merging sequences from the left and right loops. The training was performed only on the 46835 sequences with most counts, taken as unique examples (multiple copies of the same aptamer are neglected during training). The parameters are: 20 visible units, 100 hidden units, λ₁₂=0.01, 4 monte-carlo steps for each update of the parameters, mini-batches of size 200.
RBM-S+C (FIG. 4), trained on the single aptamers (20 nucleotides) obtained from the SELEX 8th round, neglecting sequences with less than 3 counts and then merging sequences from the left and right loops. The training was performed only on the 46835 sequences that, after the error correction procedure performed with the algorithm described in Sec. 4.2, had a number of inferred counts greater than zero, each taken as a unique example (multiple copies and inferred counts of each aptamer are neglected during training). The parameters are: 20 visible units, 100 hidden units, λ₁₂=0.01, 6 monte-carlo steps for each update of the parameters, mini-batches of size 100.

All the parameters for the training which are not given here are the default parameters as defined in the code.

As a final remark, we checked that the results obtained here depends very poorly on the precise values of the hyperparameters used here.

### 4.2 Error Correction Algorithm

In order to test the robustness of RBMs prediction on sequencing error we introduce an algorithm to correct for this kind of errors in our dataset. The algorithm is based on a maximum-likelihood approach and combines information on the observed copy number and relative hamming distance between different sequences to infer the most likely real copy number of all sequences in the sample. Sequences with inferred copy number equal to zero are likely to be the result of sequencing error. Here we describe this algorithm in detail. It has been validated on artificial and experimental data.

In general our maximum likelihood approach works by defining the probability of the direct process, i.e. the probability that starting from some pool of sequences in which each different sequence appears with a particular frequency, a given set of reads is obtained with a faulty reading procedure. This probability can then be inverted using Bayes theorem to obtain the likelihood of the original frequencies as a function of the set of reads. Finally, the likelihood function can be used to obtain a maximum-likelihood estimate (MLE) for the real frequency of sequences in the original sample.

### Derivation of the Probability of the Direct Process

We can frame our inference problem in the following terms. Let us consider a pool of DNA sequences of length L. Each sequence σ={σ₁, σ₂, . . . , σ_{L}} is present with a frequency f_{σ} in the pool. We call f={f_{σ}}_{σ} the set of all frequencies. A small subset of N sequences is extracted from the pool and sequenced. If this subset is much smaller than the total size of the pool then one can consider all extractions to be independent and the probability of picking sequence σ in the subset to be equal to its frequency in the pool p(σ)=f_{σ}.

The sequencing procedure is subject to errors. These can occur in the following manner: when reading the nucleotide b the apparatus can return as reading outcome nucleotide a with probability Θ[a/b]. The error probability matrix Θ will in general depend on the sequencing technology used. Here for simplicity we consider this probability to be uniform along the sequence and for all nucleotides, and to be described by just one parameter E, defined as the probability of mis-reading any given nucleotide. Notice however that the theory works for any value of the error matrix. Incorporating prior information on this matrix when available can indeed improve the inference results. In this simplification the error matrix reads:

$$Θ [ a \vert b ] = \{ \begin{matrix}1 - ϵ & if a = b \\ ϵ / 3 & if a \neq b\end{matrix} \qquad ( 11 )$$

The parameter E will be the only parameter of our algorithm, and its value should be set so as to be similar to the expected sequencing error probability. From this it follows that the probability of obtaining the read σ′ when sequencing σ is:

$${P}_{R} ( {σ}^{′} \vert σ ) = {\prod }_{l = 1}^{L} Θ [ {σ}_{l}^{′} \vert {σ}_{l} ] = {( 1 - ϵ )}^{L - {d}_{H} ( {σ}^{′} , σ )} {( ϵ / 3 )}^{{d}_{H} ( {σ}^{′} , σ )} \qquad ( 12 )$$

where d_{H}(σ′, σ) is the Hamming distance of the two sequences. Notice how “spatial” information on the sequence set is encoded in the function P_{R}(σ′/σ): the probability of mis-reading σ to σ′ is higher when the Hamming distance between the two sequences is small.

The total probability of obtaining sequence σ′ as outcome of the sequencing procedure is equal to the sum over all possible sequences σ of the probability of picking sequence σ from the pool, times the probability of obtaining read σ′ when sequencing σ:

$${P}_{read} ( {σ}^{′} \vert f ) = {\sum }_{σ} p ( σ ) {P}_{R} ( {σ}^{′} \vert σ ) = {\sum }_{σ} {f}_{σ} {P}_{R} ( {σ}^{′} \vert σ ) \qquad ( 13 )$$

The outcome of the sequencing procedure will return a number of reads no for all possible sequences σ′. This outcome is therefore completely described by the set of all counts n={n_{σ′}}_{σ′}. From our assumptions it follows that the log-probability of a given outcome n can be expressed as a function of the frequency of sequences in the pool f as:

$$log P ( n \vert f ) = {\sum }_{{σ}^{′}} {n}_{{σ}^{′}} log {P}_{read} ( {σ}^{′} \vert f ) = {\sum }_{{σ}^{′}} {n}_{{σ}^{′}} log {\sum }_{σ} {f}_{σ} {P}_{R} ( {σ}^{′} \vert σ ) \qquad ( 14 )$$

### Maximum-Likelihood Estimate for the Frequencies

Using Bayes theorem the probability derived in the previous section can be inverted to obtain the likelihood of the set of frequencies f as a function of the number of reads n. By using an uniform prior on frequencies one obtains:

$$log ℒ ( f \vert n ) = log P ( n \vert f ) + const \qquad ( 15 )$$

Where the constant term contains the normalization and does not depends on the frequencies. The MLE for the frequencies f is obtained by maximizing this likelihood under the set of constrains:

$${\sum }_{σ} {f}_{σ} = 1 \qquad ( 16 )$$

$$∀ σ , {f}_{σ} \geq 0 \qquad ( 17 )$$

Constrained optimization problems are in general hard to solve. In our case however the solution to this problem can be easily found thanks to two properties. The first is that eqs. (16) and (17) define a convex optimization domain. The second requires writing the second derivative of the log-likelihood w.r.t. the frequencies:

$$\frac{\partial }{\partial {f}_{α}} log ℒ = {\sum }_{{σ}^{′}} {n}_{{σ}^{′}} \frac{{P}_{R} ( {σ}^{′} \vert α )}{{P}_{read} ( {σ}^{′} \vert f )} \qquad ( 18 )$$

$$\frac{\partial }{\partial {f}_{α} \partial {f}_{β}} log ℒ = - {\sum }_{{σ}^{′}} {n}_{{σ}^{′}} \frac{{P}_{R} ( {σ}^{′} \vert α ) {P}_{R} ( {σ}^{′} \vert β )}{{( {P}_{read} ( {σ}^{′} \vert f ) )}^{2}} \qquad ( 19 )$$

From eq. (19) it follows that for any value of the frequencies in the domain −∂_{α,β}², log L>0, i.e. minus the log-likelihood is a convex function. Therefore our problem belongs to the class of convex optimization and can be solved numerically using gradient descent techniques.

### Numerical MLE Evaluation

Here we shortly present the algorithm we use for the numerical solution of the likelihood maximization problem. This algorithm takes as input the set of unique sequences {σ}_{σ}, the vector containing their copy number n={n_{σ}}_{σ}, and the value of the error parameter E, and will return the value of the MLE for the frequencies f={f_{σ}}_{σ}. From this the inferred value of counts can be obtained by simply \hat{n}_{σ}=N f_{σ}, where N=Σ_{σ} n_{σ} is the total number of reads in the input dataset.

Starting from an initial guess f for the MLE estimate, the algorithm works by iteratively evaluating the log-likelihood gradient Δf=∂ log L using eq. (18), normalizing it, projecting it on the subspace defined by the constraint, and updating the guess f←f+sΔf, where s is the magnitude of the update step. This magnitude is adaptively controlled and is decreased as the numerical guess for the MLE approaches the real solution, so as to increase the precision. The iteration cycle stops when the update steps reaches a sufficiently small magnitude, indicating that the real solution has been reached with sufficient numerical precision.

Here we describe more in detail every single part of the procedure. The initial guess for the frequency vector is set equal to the frequency of each sequence in the input dataset f_{σ}=n_{σ}/N. The initial magnitude of the update step to s=0.02 and the threshold precision for the update step to sₜₕᵣ=1/(1000 N), so that at convergence any successive update in frequency would change the corresponding inferred number of counts of less than 10⁻³.

The normalization and projection of the gradient Δf is performed in the following manner. First, in order to fulfill the normalization constraint eq. (16), we set Δf←Δf−(Σ_{σ} Δf_{σ})/S, where S is the total number of different sequences in the dataset S=|{σ}_{σ}|. This makes it so that Σ_{σ} Δf_{σ}=0. Moreover if for any component Δf_{σ}<0 and f_{σ}=0, we set Δf_{σ}←0, so as to respect the non-negativity constraint eq. (17), and repeat these two steps until no more components of Δf are set to zero. This provides the correct direction for the gradient in the frequency subspace. We symbolize this projection with Δf←Π(Δf, f).

This gradient is then normalized using the 2-norm Δf←Δf/∥Δf∥2, and the magnitude of the update step is given by the parameter s, so that f←f+s Δf. In some cases this might set some components of the frequencies to values <0, so the constraints given by eqs. (16) and (17) are enforced on f.

The magnitude of the update step is an important factor in controlling the convergence speed: a big value of s guarantees a quick approach to the correct solution, but a low final precision. Conversely, small values of s allow for precise recovery of the MLE, but require a longer time to convergence. As a solution we adaptively control the value of s, diminishing it as we approach the correct solution. In practice this is done by keeping a record of the value of the log-likelihood at every iteration of the algorithm. This value is saved in a vector Lᵢ, where i indicates the iteration index. Every 20 iterations the following check is performed: the magnitude of the total increase in log-likelihood |Lᵢ−Li−20| is compared to the sum of magnitudes of single-iteration variations Σⱼ₌ᵢ₋₂₀ⁱ⁻¹ Lⱼ₊₁−Lⱼ|. The adaptive step is halved s←s/2 if the following condition is detected:

$$2 \vert {L}_{i} - {L}_{i - 20} \vert < {\sum }_{j = i - 20}^{i - 1} \vert {L}_{j + 1} - {L}_{j} \vert \qquad ( 20 )$$

The idea is that when the correct solution is approached within a precision comparable to s then successive updates will not result in a likelihood decrease, but rather in an oscillation of the value of Lᵢ. The above comparison picks up this oscillation and reduces the magnitude of the update step s, thus allowing for a more precise convergence until at sufficient precision a new oscillation state is reached.

Finally, when the value of the adaptive step magnitude becomes smaller than the threshold s<sₜₕᵣ then convergence is reached, the iteration is stopped and the current value of the frequency vector f is returned.

A simplified pseudocode for the procedure described is reported in Algorithm 1.

Algorithm 1: Maximum-Likelihood frequency estimation

| | |
|---|---|
| input : | list of observed sequences {σ}_{σ}, |
| | copy number for each sequence n = {n_{σ}}_{σ}, |
| | error rate ϵ. |

output: Maximum likelihood estimate of the frequency f = {f_{σ}}_{σ},
Evaluate the total number of reads N ← Σ_{σ} n_{σ};
Initialize frequency vector f with initial guess f_{σ} ← n_{σ}/N;
Set convergence threshold sₜₕᵣ ← 1/(1000N);
Initialize the adaptive update step to s ← 0.02 ;
Initialize the iteration number i ← 0;
Evaluate log-likelihood Lᵢ ← log L(f) using eq. (15);
set convergence state c ← false
while not c do
| Update iteration number i ← i + 1;
| Evaluate the log-likelihood gradient Δf ← δlog L(f) using eq. (18);
| Project Δf ← II(Δf, f) on constraints and normalize it so that ||Δf||₂ = 1;
| Update f ← f + s Δf, set negative components to zero and normalize f;
| Evaluate log-likelihood Lᵢ ← log L(f) using eq. (15);
| if oscillation on L is detected (cf. eq 20) then
| | update the adaptive step s ← s/2;
| | if s < sₜₕᵣ then
| | |_ set convergence state to true c ← true;
| |_
|_
return frequency vector f

TABLE 5

| Model | Description |
|---|---|
| Long Resnet | A 152 layer Resnet followed by a dropout layer, a 512 input to 2 output linear layer |
| | followed by a softmax layer. Residual networks guarantee performance of subsequent |
| | layers in the network by mapping to a residual function F(x) = H(x)-x. This network |
| | architecture has been shown to avoid vanishing gradients and accuracy degradation |
| | present in traditional network architecture learning. During training, this model used label |
| | smoothed (smoothing = 0.01) cross entropy as its loss function. |
| Short Resnet | An 18 layer Resnet followed by a dropout layer, a 512 input to 512 output linear layer, a |
| | naively implemented DReLU activation function, a 512 input to 2 output linear layer, and |
| | finally a softmax layer. During training, this model used label smoothed (smoothing = 0.01) |
| | cross entropy as its loss function. |
| Long Variational Auto Encoder | A 152 layer Resnet followed by two 2 encoder blocks encoded the embedding. The |
| | decoder consisted of 4 decoder blocks. Encoder blocks consisted of a spectral normalized |
| | 2d convolution layer, followed by 2d batch normalization and a leaky ReLU activation |
| | function. Likewise decoder blocks consisted of a transposed 2d convolution followed by 2d |
| | batch normalization and a leaky ReLU activation function. Self-attention layers were added |
| | in between both encoder and decoder blocks. Variational AutoEncoders are generative |
| | models designed to sample across a continuous latent space. Each embedding was also |
| | trained on a 512 input to 2 output linear layer connected to a log softmax layer to generate |
| | a binding/nonbinding verdict. During training this model used label smoothed |
| | (smoothing = 0.01) cross entropy on the predictions and symmetric MSE loss on the |
| | decoder's reconstruction. The loss functions were mixed for the total training loss. |
| Short Variational | A 152-layer Resnet encoder and 2 decoder block, separated by an attention layer. A 512 |
| Auto Encoder | input to 2 output linear layer was trained on each embedding with a log softmax layer on |
| | the end to predict a binding vs. nonbinding result. During training this model used label |
| | smoothed (smoothing = 0.01) cross entropy on the predictions and symmetric MSE loss on |
| | the decoder's reconstruction. The loss functions were mixed for the total training loss. |
| Siamese | A Siamese network trained on pairs of sequences to discriminate between binder-binder |
| | pairs and nonbinder-binder pairs. The Siamese network used here consisted of a single |
| | resnet made up of 4 layers with 512 input to 256 output linear layer, a sigmoid activation |
| | function, and a 256 input to 2 output linear layer following. Each iteration was run |
| | individually on pairs of sequences. The Euclidean distance between the resulting |
| | embeddings is used to assign our binary classification value. During training this model |
| | used contrastive loss as its loss function. |

Some further aspects are defined in the following clauses:

Clause 1: A method of generating a trained classifier at least partially using a computer, the method comprising training, by the computer, a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model, thereby generating the trained classifier at least partially using the computer.

Clause 2: The method of Clause 1, comprising applying a maximum likelihood algorithm to the first training dataset to identify and exclude erroneous information.

Clause 3: The method of Clause 1 or Clause 2, wherein the first training dataset comprises one or more sequence motifs.

Clause 4: The method of any one of the preceding Clauses 1-3, comprising repeating the training step using at least a second training dataset.

Clause 5: The method of any one of the preceding Clauses 1-4, wherein the target biomolecule comprises thrombin.

Clause 6: The method of any one of the preceding Clauses 1-5, comprising generating candidate aptamer sequence information using the trained RBM model.

Clause 7: The method of any one of the preceding Clauses 1-6, comprising synthesizing the candidate aptamer using the candidate aptamer sequence information to produce a synthesized candidate aptamer.

Clause 8: The method of any one of the preceding Clauses 1-7, comprising using the synthesized candidate aptamer to bind the target biomolecule.

Clause 9: A method of generating a candidate aptamer, the method comprising: generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule; and, synthesizing the candidate aptamer using the candidate aptamer sequence information, thereby generating the candidate aptamer.

Clause 10: The method of Clause 9, wherein the trained RBM model is generated at least in part by applying a maximum likelihood algorithm to the first training dataset to identify and exclude erroneous information.

Clause 11: The method of Clause 9 or Clause 10, wherein the first training dataset comprises one or more sequence motifs.

Clause 12: The method of any one of the preceding Clauses 9-11, wherein the target biomolecule comprises thrombin.

Clause 13: The method of any one of the preceding Clauses 9-12, comprising using the synthesized candidate aptamer to bind the target biomolecule.

Clause 14: A system, comprising a controller comprising, or capable of accessing, computer readable media comprising non-transitory computer executable instructions which, when executed by at least one electronic processor, perform at least training a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model.

Clause 15: The system of Clause 14, wherein the executable instructions which, when executed by the electronic processor, further perform at least: applying a maximum likelihood algorithm to the first training dataset to identify and exclude erroneous information.

Clause 16: The system of Clause 14 or Clause 15, wherein the first training dataset comprises one or more sequence motifs.

Clause 17: The system of any one of the preceding Clauses 14-16, wherein the executable instructions which, when executed by the electronic processor, further perform at least: repeating the training step using at least a second training dataset.

Clause 18: The system of any one of the preceding Clauses 14-17, wherein the target biomolecule comprises thrombin.

Clause 19: The system of any one of the preceding Clauses 14-18, wherein the executable instructions which, when executed by the electronic processor, further perform at least: generating candidate aptamer sequence information using the trained RBM model.

Clause 20: The system of any one of the preceding Clauses 14-19, wherein the executable instructions which, when executed by the electronic processor, further perform at least: synthesizing the candidate aptamer using the candidate aptamer sequence information and an operably connected a biomolecule synthesis device to produce a synthesized candidate aptamer.

Clause 21: A system, comprising: a biomolecule synthesis device; and at least one controller operably connected to the biomolecule synthesis device, which controller comprises, or is capable of accessing, computer readable media comprising non-transitory computer executable instructions which, when executed by at least one electronic processor, perform at least: generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule; and, synthesizing the candidate aptamer using the biomolecule synthesis device and the candidate aptamer sequence information.

Clause 22: The system of Clause 21, wherein the trained RBM model is generated at least in part by applying a maximum likelihood algorithm to the first training dataset to identify and exclude erroneous information.

Clause 23: The system of Clause 21 or Clause 22, wherein the first training dataset comprises one or more sequence motifs.

Clause 24: The system of any one of the preceding Clauses 21-23, wherein the target biomolecule comprises thrombin.

Clause 25: A computer readable media comprising non-transitory computer executable instruction which, when executed by at least electronic processor perform at least training a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model.

Clause 26: The computer readable media of Clause 25, wherein the executable instructions which, when executed by the electronic processor, further perform at least: applying a maximum likelihood algorithm to the first training dataset to identify and exclude erroneous information.

Clause 27: The computer readable media of Clause 25 or Clause 26, wherein the first training dataset comprises one or more sequence motifs.

Clause 28: The computer readable media of any one of the preceding Clauses 25-27, wherein the executable instructions which, when executed by the electronic processor, further perform at least: repeating the training step using at least a second training dataset.

Clause 29: The computer readable media of any one of the preceding Clauses 25-28, wherein the target biomolecule comprises thrombin.

Clause 30: The computer readable media of any one of the preceding Clauses 25-29, wherein the executable instructions which, when executed by the electronic processor, further perform at least: generating candidate aptamer sequence information using the trained RBM model.

Clause 31: The computer readable media of any one of the preceding Clauses 25-30, wherein the executable instructions which, when executed by the electronic processor, further perform at least: synthesizing the candidate aptamer using the candidate aptamer sequence information and an operably connected a biomolecule synthesis device to produce a synthesized candidate aptamer.

Clause 32: A computer readable media comprising non-transitory computer executable instruction which, when executed by an electronic processor perform at least: generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule; and, synthesizing a candidate aptamer using an operably connected biomolecule synthesis device and the candidate aptamer sequence information.

Clause 33: The computer readable media of Clause 32, wherein the trained RBM model is generated at least in part by applying a maximum likelihood algorithm to the first training dataset to identify and exclude erroneous information.

Clause 34: The computer readable media of Clause 32 or Clause 33, wherein the first training dataset comprises one or more sequence motifs.

Clause 35: The computer readable media of any one of the preceding Clauses 32-34, wherein the target biomolecule comprises thrombin.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, and/or computer readable media or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A method of generating a trained classifier at least partially using a computer, the method comprising training, by the computer, a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model, thereby generating the trained classifier at least partially using the computer.

2. The method of claim 1, comprising applying a maximum likelihood algorithm to the first training dataset to identify and exclude erroneous information.

3. The method of claim 1, wherein the first training dataset comprises one or more sequence motifs.

4. The method of claim 1, comprising repeating the training step using at least a second training dataset.

5. The method of claim 1, wherein the target biomolecule comprises thrombin.

6. The method of claim 1, comprising generating candidate aptamer sequence information using the trained RBM model.

7. The method of claim 1, comprising synthesizing the candidate aptamer using the candidate aptamer sequence information to produce a synthesized candidate aptamer.

8. The method of claim 7, comprising using the synthesized candidate aptamer to bind the target biomolecule.

9. The trained RBM model produced by the method of claim 1.

10. A method of generating a candidate aptamer, the method comprising:
generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule; and,
synthesizing the candidate aptamer using the candidate aptamer sequence information, thereby generating the candidate aptamer.

11. The method of claim 10, wherein the trained RBM model is generated at least in part by applying a maximum likelihood algorithm to the first training dataset to identify and exclude erroneous information.

12. The method of claim 10, wherein the first training dataset comprises one or more sequence motifs.

13. The method of claim 10, wherein the target biomolecule comprises thrombin.

14. The method of claim 10, comprising using the synthesized candidate aptamer to bind the target biomolecule.

15. A system, comprising a controller comprising, or capable of accessing, computer readable media comprising non-transitory computer executable instructions which, when executed by at least one electronic processor, perform at least training a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model.

16. The system of claim 15, wherein the executable instructions which, when executed by the electronic processor, further perform at least: applying a maximum likelihood algorithm to the first training dataset to identify and exclude erroneous information.

17. The system of claim 15, wherein the first training dataset comprises one or more sequence motifs.

18. The system of claim 15, wherein the executable instructions which, when executed by the electronic processor, further perform at least: repeating the training step using at least a second training dataset.

19. The system of claim 15, wherein the target biomolecule comprises thrombin.

20. The system of claim 15, wherein the executable instructions which, when executed by the electronic processor, further perform at least: generating candidate aptamer sequence information using the trained RBM model.

21. The system of claim 15, wherein the executable instructions which, when executed by the electronic processor, further perform at least: synthesizing the candidate aptamer using the candidate aptamer sequence information and an operably connected a biomolecule synthesis device to produce a synthesized candidate aptamer.

22. A system, comprising:
a biomolecule synthesis device; and
at least one controller operably connected to the biomolecule synthesis device, which controller comprises, or is capable of accessing, computer readable media comprising non-transitory computer executable instructions which, when executed by at least one electronic processor, perform at least:
generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule; and,
synthesizing the candidate aptamer using the biomolecule synthesis device and the candidate aptamer sequence information.

23. The system of claim 22, wherein the trained RBM model is generated at least in part by applying a maximum likelihood algorithm to the first training dataset to identify and exclude erroneous information.

24. The system of claim 22, wherein the first training dataset comprises one or more sequence motifs.

25. The system of claim 22, wherein the target biomolecule comprises thrombin.

26. A computer readable media comprising non-transitory computer executable instruction which, when executed by at least electronic processor perform at least training a Restricted Boltzmann Machine (RBM) using at least a first training dataset that comprises sequence information corresponding to a population of aptamers, and/or one or more descriptors thereof, which aptamers comprise a minimum threshold binding affinity to a target biomolecule to produce a trained RBM model.

27. The computer readable media of claim 26, wherein the executable instructions which, when executed by the electronic processor, further perform at least: applying a maximum likelihood algorithm to the first training dataset to identify and exclude erroneous information.

28. The computer readable media of claim 26, wherein the first training dataset comprises one or more sequence motifs.

29. The computer readable media of claim 26, wherein the executable instructions which, when executed by the electronic processor, further perform at least: repeating the training step using at least a second training dataset.

30. The computer readable media of claim 26, wherein the target biomolecule comprises thrombin.

31. The computer readable media of claim 26, wherein the executable instructions which, when executed by the electronic processor, further perform at least: generating candidate aptamer sequence information using the trained RBM model.

32. The computer readable media of claim 26, wherein the executable instructions which, when executed by the electronic processor, further perform at least: synthesizing the candidate aptamer using the candidate aptamer sequence information and an operably connected a biomolecule synthesis device to produce a synthesized candidate aptamer.

33. A computer readable media comprising non-transitory computer executable instruction which, when executed by an electronic processor perform at least:
generating candidate aptamer sequence information using a trained Restricted Boltzmann Machine (RBM) model produced using at least a first training dataset that comprises sequence information corresponding to a population of aptamers, and/or one or more descriptors thereof, which aptamers comprise a minimum threshold binding affinity to a target biomolecule; and,
synthesizing a candidate aptamer using an operably connected biomolecule synthesis device and the candidate aptamer sequence information.

34. The computer readable media of claim 33, wherein the trained RBM model is generated at least in part by applying a maximum likelihood algorithm to the first training dataset to identify and exclude erroneous information.

35. The computer readable media of claim 33, wherein the first training dataset comprises one or more sequence motifs.

36. The computer readable media of claim 33, wherein the target biomolecule comprises thrombin.
